(19) United States
(12) Patent Application Publication
(10) Pub. No.: US 2004/0018495 A1
(43) Pub. Date: Jan. 29, 2004

(54) Microparticle based signal amplification for the detection of analytes

(76) Inventor: Xing-Xiang Li, Vienna, VA (US)

Correspondence Address: Mark W. Roberts, Esq., DORSEY & WHITNEY LLP, Suite 3400, 1420 Fifth Avenue, Seattle, WA, 98101, US

(21) Appl. No.: 10/205,195

(22) Filed: Jul. 24, 2002

(51) Int. Cl. C12Q 1/68

(52) U.S. Cl. 435/6

(57) ABSTRACT

Microparticle based amplification (MBA) for high sensitivity and high speed analyte detection is described. MBA is based on signal amplification achieved by use of a signal amplification microparticle that contains a plurality of signaling molecules attached to a plurality of positions on the surface of the microparticle, in combination with a plurality of analyte binding molecules attached to a plurality of positions on the surface. Each signaling molecule in turn has a plurality of signal emitting moieties, such as acridinium, attached thereto. This is combined with a separating microparticle such as a ferromagnetic particle, also having an analyte binding molecule attached to the surface so that a complex comprising the analyte, the signal amplification microparticle and the separating microparticle is formed. The complex emits a signal that is amplified many fold relative to the stoichemetric amount of analyte molecules in the sample. Particular embodiments include methods for detecting bacteria, antigens, antibodies and nucleic acids.

### TECHNICAL FIELD

The invention relates to microparticles as well as kits and methods using the same for the detection of analytes, and particularly to signaling microparticles attached to a plurality of signal emitting molecules and attached to an analyte binding molecule for binding to the analyte. The methods include combinations of different types of analyte binding microparticles to facilitate rapid and sensitive detection of the analyte.

### BACKGROUND OF THE INVENTION

Detecting biological analytes such as bacteria, antigens, antibodies, receptors, ligands and nucleic acids is pivotal to diagnostic test methods for a wide variety of diseases and conditions and is important to research, forensic and risk assessment applications. Such methods typically rely on specific binding between a target biological analyte and a corresponding analyte binding molecule to form a complex that can be readily detected. For example, bacteria may be detected by binding to particular lecithins or antibodies specific for surface antigens on the bacteria. Soluble antigens may be detected by binding to specific antibodies raised against the antigen. Conversely, specific antibodies may be detected by binding to their corresponding antigens (or antigen conjugates). Receptors on cell surfaces indicative of particular cell types may be detected by binding to their corresponding ligands. Conversely, ligands may be detected by binding to their corresponding receptors. Nucleic acids may be detected by hybridizing to complementary nucleic acid sequences. Central to all these detection methods is the ability to detect the formation of a bound complex between the target analyte and the analyte binding molecule, which is distinguishable from non-complexed molecules. Typically the bound complex is detected by one of three basic techniques.

The first basic technique relies on a change in the physical state of the analyte binding molecule complexed with the analyte relative to either component alone. A common example of this technique is antibody agglutination, whereby an antibody or an antibody bound to a particle, such as a latex particle, becomes interconnected through cross binding to form a lattice of sufficient size to scatter light in comparison to non-analyte bound antibodies. A common form of an agglutinating assay relies on adhering an antibody to the surface of a microtiter plate and contacting the same with a sample containing the analyte and a reagent having a second antibody linked to latex particles. After a sufficient period of incubation, a light scattering lattice comprised of the latex particles complexed with the analyte forms on the surface of the microtiter plate, and the size of the lattice is measured by light scattering or absorption. One disadvantage of agglutinating assays and similar physical state based methods is that they are relatively insensitive, requiring a relatively large amount of analyte material to reliably detect the complex.

The second basic technique for detecting an analyte complex is the ELISA method, which relies on linking an enzyme to an antibody. The enzyme linked species forms a sandwich complex with the analyte and another antibody (or antigen) species typically immobilized on a surface. After washing the surface bound complex to remove unbound enzyme-linked molecules, the bound complex is incubated with a substrate for the enzyme to detect the conversion of the substrate to a product that is measured by conventional spectrophotometeric or chemoluminescent techniques. ELISA methods provide the benefit of relatively high sensitivity, but have the disadvantage of taking a relatively long time to execute to obtain maximum sensitivity. In a typical ELISA test, the antibody-antigen binding may require several hours (typically overnight) to reach equilibrium (100% completion) while a 10-minute incubation might reach only about 3% completion. ELISA test are therefore not practical for rapid on site testing in a short time frame, such as for rapid screening patients or blood donors for diseases. ELISA tests also have other disadvantages such as instability of the linked enzyme, relatively expensive substrates and requiring multiple steps to execute, all of which lead to relatively high costs for ELISA tests.

The third basic analyte complex detection technique is labeling, which relies on detecting a label attached to the analyte binding molecule after it is bound to the analyte. Typically the analyte sample is immobilized on a substrate, incubated with the labeled analyte binding molecule, and then washed to remove unbound labeled molecules. Labeling techniques are most commonly used with nucleic acid detection methods where the analyte binding molecule is a nucleic acid probe that is hybridized to a complementary sequence of the target analyte nucleic acid. A variety of label types have been used in this regard, including for example, radioactive, fluorescent, chemiluminescent and electroluminescent species. A variety of substrates have also been used, from simple filter-like membranes to complex nucleic acid chip arrays. In the clinical arena, the most commonly used nucleic acid binding tests are for screening blood for viruses (e.g., HIV, HCV and HBV) or for HIV viral load testing. Viral load tests are used to measure viral concentration in the plasma as a means to monitor effectiveness of anti-viral drug therapy or disease progression. Nucleic acid labeling techniques are frequently used in conjunction with electrophoresis or other size separating procedures to identify target analyte molecules of particular sizes.

One of the major disadvantages of conventional labeling techniques, especially those associated with detecting nucleic acids is that they are relatively complex, requiring skill and training to execute. Another disadvantage is that like ELISA tests, labeling test require a relatively long incubation period to drive the hybridization to a sufficient level to detect the label. Another related disadvantage is that the amount of label attached to the probe is limited by the size of the probe and the necessity of protecting the hydrogen bonding domains for hybridization. This limits the sensitively of detection, which is sometimes addressed by analyte amplification techniques such as PCR (polymerase chain reaction) to amplify the target analyte nucleic acid. PCR adds another level of complexity (and variability) associated with the enzymes, reagents and protocols needed for reliable PCR. Yet another disadvantage is that relatively expensive equipment is required for the most sensitive types of detection. These disadvantages effectively proscribe the use of conventional labeling techniques from applications requiring rapid and inexpensive on-site detection at the time of service, such as required in the clinical arena.

Accordingly, there is a need in the art for compositions and methods for improving the sensitivity, speed and simplicity of analyte detection, and especially for such compositions and methods that are readily adaptable for detecting a wide variety of analytes.

### SUMMARY OF THE INVENTION

Described herein are compositions and methods designated microparticle based amplification (MBA) for increasing the speed, sensitivity and simplicity of analyte detection and which is adaptable to wide variety of techniques. With MBA, as little as 3% of an analyte binding molecule bound to an analyte in a one mL reaction can be detected in a 10 to 20 minute protocol. Methods based on MBA technology involve three basic steps: binding of two types of microparticles to an analyte, i.e., a signal amplification microparticle and a separating microparticle, separation of the dual bound microparticle complex from non-complexed signal amplification microparticles, and detection of a light signal emitted form one of the microparticles. The speed and cost for labor and reagents is significantly lower than that for other analyte detection technologies.

MBA is based on signal amplification achieved by use of the signal amplification microparticle, which contains a plurality of signaling molecules attached to a plurality of attachment positions on the surface of the microparticle, in combination with plurality of analyte binding molecules also attached to a plurality of positions on the surface. Each signaling molecule in turn has a plurality of signal emitting moieties attached thereto. The methods combine the signal amplification microparticle with the separating microparticle, which has physical property different than the first microparticle that permits the separating microparticle to be readily separated from the signal amplification microparticle. The separating microparticle also has an analyte binding molecule attached to the surface. Therefore, a mixture containing the analyte, the signal amplification microparticle and the separating microparticle forms a complex where the signal amplification microparticle is bound to the analyte, which is also bound to the separating microparticle. The complex may be formed in stepwise fashion in which the analyte is first bound to the separating microparticle to form a primary complex, which after separated from unbound analyte, binds to the signal amplification microparticle to form a final complex. The bound complex is readily separated from unbound signal amplification microparticles in accordance with the separation property of the separating microparticle. When separated, the signal detected from the complex directly corresponds to the amount of signal amplification microparticles in the complex, which in turn corresponds to the amount of analyte in the sample. The detected signal is amplified many fold relative to the stoichemetric amount of analyte molecules in the sample because the analyte molecule is bound to a signal amplification microparticle that has multiple signaling molecules, each with multiple signal emitting moieties.

The signaling molecule on the signal amplification microparticle comprises a polymer, which has a domain that conjugates to the microparticle (i.e., a first conjugation domain) and a signal binding domain that contains a plurality of functional groups linking to the plurality of signal emitting moieties. In certain embodiments, the signaling molecule is an oligonucleotide where the first conjugation domain has a sequence complementary to a corresponding attachment oligonucleotide moiety on the surface of the signal amplification microparticle. The signal binding domain includes sequence with a plurality of derivatized amine groups that are used to link to a plurality of acridinium moieties. In various embodiments the analyte binding molecule may be an antibody, antigen, receptor, ligand or nucleic acid. The analyte binding molecule has an analyte binding domain and also has a domain for conjugation to the surface of the microparticle (i.e., a second conjugation domain). In certain embodiments, the second conjugation domain includes a conjugated oligonucleotide having a sequence complementary to a second oligonucleotide attachment moiety on the surface of the signal amplification microparticle. In alternative embodiments, the signaling molecule and/or the analyte binding molecule may be conjugated to the signal amplification microparticle by direct cross linking.

The separating microparticle may be conjugated to the second analyte binding molecule in a like manner as the first analyte binding molecule is conjugated to the signal amplification microparticle. The second analyte binding molecule may therefore include an oligonucleotide conjugation domain (i.e., a third conjugation domain) with a sequence complementary to a third oligonucleotide attachment moiety present on the surface of the separating microparticle. The second and third oligonucleotide attachment moieties may have the same sequence or different sequences. The second analyte binding molecule may be the same or different type from the first analyte binding molecule. Alternatively, the second analyte binding molecule may be conjugated to the separating microparticle by direct cross linking.

Certain embodiments include compositions where the first and second analyte binding molecules are antibodies that bind to antigens, particularly antigens on the surface of bacteria. Other embodiments include compositions where the first and second analyte binding molecules are antigens that bind to antibodies present in a sample, and in particular embodiments, to HIV antibodies. Still other embodiments include compositions where the first and second analyte binding molecules are nucleic acids having a first and second sequences, respectively, which are complementary to first and second target sequences of a target nucleic acid analyte.

Kits and methods based on MBA are also described. In certain embodiments the kits include the signal amplification microparticle conjugated to a particular antibody analyte binding molecule and to a signaling oligonucleotide molecule containing a plurality of acridinium moieties attached thereto. The separating microparticle is a magnetic particle conjugated to the same type of antibody analyte binding molecule. The methods include using such compositions and/or kits to detect an analyte by forming a mixture containing the signal amplification microparticle, the separating microparticle, and the analyte, incubating the mixture for a period of time sufficient for the first and second analyte binding molecules to bind the analyte, partitioning the mixture into a sample containing the separating microparticle using a magnet, washing the separated microparticles, exposing the washed microparticles to conditions that cause light to be emitted from the attached signal amplification microparticles, and detecting the emitted light signal.

The methods are suitable for assays conducted in 10 to 20 minutes using a relatively inexpensive and portable luminometer. A bacterial detection system utilizing such molecules in combination with a luminometer is suitable for use in a point-of-care (POC) setting such as hospitals, at least in part, because an assay can be completed within 15-30 minutes with a high level of confidence and sensitivity. Therefore, the test may be used, for example, in testing platelets for bacteria contamination just prior to a transfusion procedure. In particular embodiments, the design sensitivity (limit of detection) is 5000 bacteria/mL or better for at least five bacterial strains. Typical procedures take 10 to 20 minutes for the binding reaction, 4-5 minutes for washing (rinsing) and 1 minute for the chemiluminescence's reaction.

The speed, sensitivity and lack of complexity using the compositions and methods described herein make such methods suitable for easy automation and for handling large number of samples.

### BRIEF DESCRIPTION OF THE DRAWINGS

FIG. 1 depicts a signal amplification microparticle according to one aspect of the invention. FIG. 1A illustrates the core of the signal amplification microparticle and FIG. 1B illustrates an embodiment of a complete signal amplification microparticle.

FIG. 2 depicts one embodiment of the signal amplification microparticle according to the invention.

FIG. 3A depicts an aspect of the signal amplification microparticle of the invention. FIG. 3B depicts another aspect of the signal amplification microparticle of the invention. FIG. 3C depicts yet another aspect of the signal amplification microparticle of the invention. FIGS. 3D to 3G exhibit an aspect of the signal amplification microparticle of the invention where the signal emitting molecule is replaced with a signal generating molecule, e.g., alkaline phosphatase, which itself does not emit signal but catalyzes the formation of a signal. FIGS. 3D to 3G depicts various means by which a signal generating molecule is attached to a microparticle.

FIG. 4 depicts a kit using the signal amplification microparticle and a separating microparticle according to another aspect of the invention. FIG. 4A depicts the kit with one embodiment of the separating microparticle and FIG. 4B depicts another embodiment of the microparticle.

FIG. 5 is a flow chart illustrating a method according to another aspect of the invention. FIG. 5A depicts one procedure involving a one-step complex formation process and FIG. 5B depicts an alternative procedure involving a two-step complex formation process.

FIG. 6 illustrates detecting a bacterial analyte according to the invention.

FIG. 7 illustrates one embodiment for detecting an antibody analyte according to the invention.

FIG. 8 illustrates another embodiment for detecting an antibody analyte according to the invention.

FIG. 9 illustrates yet another embodiment for detecting an antibody analyte according to the invention.

FIG. 10 illustrates detecting a nucleic acid analyte according to the invention.

FIG. 11 depicts another embodiment of the signal amplification microparticle and separating microparticle of the invention.

FIG. 12 illustrates oligonucleotides used in assembling the signal amplification microparticle according to the invention. FIG. 12A illustrates oligonucleotides A-D that are used in certain embodiments, and FIG. 12B illustrates assembly of the oligonucleotide C, which is used for the signal molecule in certain embodiments.

FIG. 13 illustrates the expected distribution patterns of test results of negative sample replicates. The cutoff RLU value can be set according to the mean and standard deviation (SD). Here, the cutoff RLU is set at 3 SDs above the mean.

FIG. 14 illustrates determination of a desired sensitivity for detecting bacteria according to the invention.

FIG. 15 illustrates one method for determination of antibody titer.

### DETAILED DESCRIPTION OF THE INVENTION

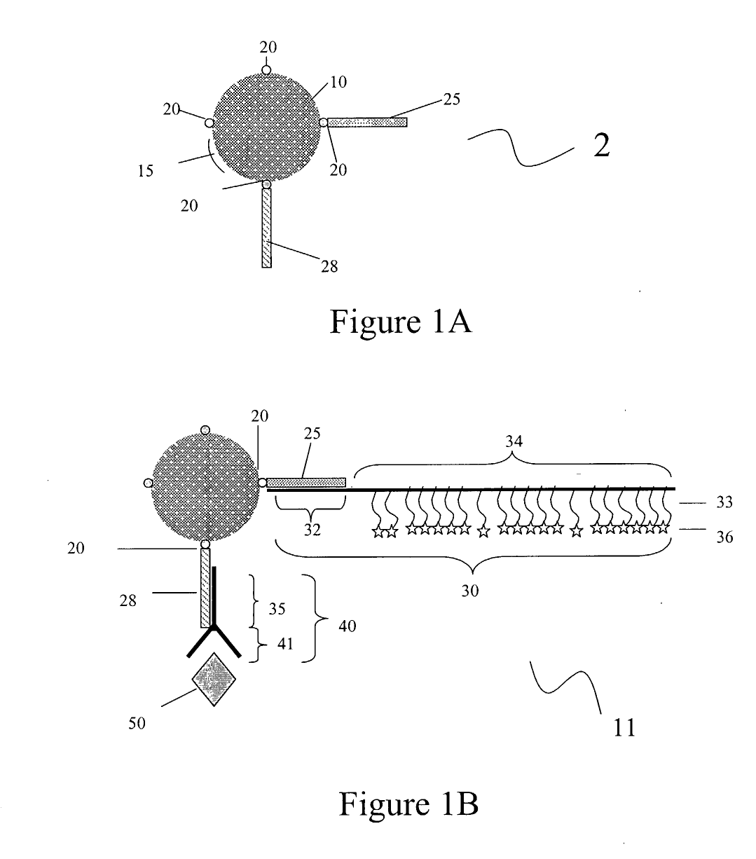

FIG. 1 illustrates general features of a signal amplification microparticle 11 used for MBA according to one aspect of the present invention. The signal amplification microparticle 11 is assembled from a core microparticle 2 depicted in FIG. 1A. The core microparticle 2 includes a first microparticle 10 having a surface 15 with a plurality of functional groups 20 to which a first attachment moiety 25 and a second attachment moiety 28 are bound. The first attachment moiety 25 and the second attachment moiety 28 are typically bound to the first microparticle 10 by covalently cross-linking the functional groups 20 on the first microparticle 10 with functional groups present on the first and second attachment moieties 25 and 28.

FIG. 1B illustrates the core microparticle 2 assembled to form the signal amplification microparticle 11. The signal amplification microparticle 11 includes a signal molecule 30 bound to the surface of the first microparticle 10 through the first attachment moiety 25. The signal molecule includes a conjugation domain (i.e., a first conjugation domain 32) that binds to the first attachment moiety 25 and a signal binding domain 34 that binds to a plurality of signal emitting moieties 36. The signal amplification microparticle 11 is also bound to an analyte binding molecule 40 through the second attachment moiety 28. The analyte binding molecule 40 also includes a conjugation domain (i.e., the second conjugation domain 35) that binds to the second attachment moiety 28 and has a first analyte binding domain 44 that binds the analyte 50. As described in more detail hereafter, suitable analyte binding molecules 40 include, but are not limited to, antigens, antibodies, proteins, receptors and ligands, where the corresponding analyte would be an antibody, antigen, substrate, ligand or receptor, respectively. In some embodiments, the first conjugation domain 32 and/or the second conjugation domain 35 are bound to the first and second attachment moieties 25 and 28, respectively, by non-covalent bonding such as hydrogen bonding between complementary nucleic acid sequences. In other embodiments, the first conjugation domain 32 and/or the second conjugation domain 35 are bound to the first and second attachment moieties 25 and 28, respectively, by covalent bonds formed by chemical cross linking of functional groups on the attachment moieties 25 and 28 with functional groups on the respective conjugation domains 32 and 35. In certain embodiments, the first conjugation domain 32 and/or the second conjugation domain 35 are directly attached microparticles via covalent bonds.

The signal molecule 30 may be any polymeric species that includes the conjugation domain 32 having a molecular structure that can be conjugated to the first attachment moiety 25, and which has the signal binding domain 34 that includes a plurality of functional groups 33 that can be conjugated to the plurality of signal emitting moieties 36. As used herein, “conjugation” means any chemical binding between different molecular species including non-covalent and covalent binding. In a typical embodiment, the conjugation domain 32 is conjugated to the attachment moiety by hydrogen bonding and the signal binding domain 34 is conjugated to the signal emitting moiety 36 by covalent bonding through functional groups 33. Example species for the signal binding domain 34 of the signaling molecule 30 include, but are not limited to, polymers containing a plurality of amine groups such as polynucloetides, polylysine, polyarginine, polyglutamine, polyhistidine, poly-amino-saccharides, spermine, spermidine, and polypeptides having a plurality of amine side chain functional groups. Other example species for the signal binding domain 34 of the signaling molecule 30 include, but are not limited to, polymers containing polycarboxylic acid groups such as polyglutamate, polyasparte, polyglyconates and polypeptides having a plurality of carboxyl side chain functional groups. Poly-amine and poly-carboxyl containing polymers are preferred species for the signal binding domain 34 of the signaling molecule 30 because cross-linking chemistry for linking these types of functional groups 33 with the most common functional groups present on most signal emitting moieties 36 is well known in the art. However, any polymer having a plurality of functional groups 33 that can be cross linked to the signal emitting moiety 36 is suitable.

Oligonucleotides of defined sequence containing nucleotides derivatized with functional groups, or their functionally homologous chemicals, are preferred for the signal binding domain 34 of the signal molecule 30 because these functional groups provide convenient vehicles for chemical cross linking to the signal emitting moiety 36. Oligonucleotides of defined sequence are also preferred for the conjugation domain 32 of the signal emitting molecule 30 because oligonucleotides can be readily conjugated to the first attachment moiety 25 by hybridization of complementary sequences. In mixed embodiments of the signal emitting molecule 30, the conjugation domain 32 may be comprised of an oligonucleotide sequence while the signal binding domain 34 may be comprised of a non-nucleotide polymer such as polylysine or polyglutamate. Techniques for covalently cross linking oligonucleotides to functional groups on non-oligonucleotide polymers are well known in the art. Accordingly, the signal molecule 30 may be formed from a large variety of chemical species so long as it contains at least one functional group residue to serve as the conjugation domain 32 and a plurality of functional groups 33 to serve as the signal-binding domain 34.

The signal emitting moiety 36 may be any chemical moiety that emits a signal which is detectable by an appropriate instrument. In typical embodiments, the signal emitting moiety emits light, which is readily detectable by a luminometer. In certain embodiments the signal emitting moiety 36 emits light as a result of being exposed to particular chemical or physical conditions, for example, as a result of a chemical reaction with a reagent that changes the oxidation/reduction environment of the signal emitting moiety 36 such as chemiluminescent emissions, or as a result of being excited by radiant energy as in the case of fluorescent and phosphorescent light emissions, or as a result of electrical stimulation as in the case of electroluminescent emissions. Example electroluminescent materials include but are not limited to those described in U.S. Pat. Nos. 6,333,122, 6,329,083, 6,277,503, 6,271,626, 6,180,267, 6,143,434, 5,747,183 and 5,706,224, each incorporated herein by reference. One example of a chemiluminescent signal emitting moiety 36 is acridinium, which when derivatized with an ester moiety, can readily be cross linked to amines, particularly primary amines, present on the signal binding domain 34 of the signal emitting molecule 30. In the presence of an oxidizing species, such as hydrogen peroxide, superoxide acridinium produces a chemiluminescent emission that is detectable in a luminometer Another example of the signal emitting moiety 36 is fluorescein, which is a fluorescent molecule that emits fluorescent light readily detectable by excitation in a fluourimeter. Techniques for cross linking fluorescein to amines and to oligonucleotides are well known in the art. In other embodiments, the signal emitting moiety may be an enzyme such as alkaline phosphatase or peroxidase, which is used in combination with a substrate where the substrate is converted to a product that emits the signal. In certain embodiments the signal emitting moiety 36 facilitates the generation of color signals.

The first microparticle 10 of the signal amplification microparticle 11 may be made of any particulate macromolecular structure including but not limited to solid beads, porous beads, ferromagnetic beads and the like. Such beads are commonly referred to in the art as nanoparticles, microparticles, beads or microspheres. In most embodiments, the microparticle 10 used with the signal amplification microparticle 11 has an average diameter of less than about 2 μm, and preferably less than about 1 μm. In typical embodiments, the microparticle 10 has a diameter of about 0.001 μm to about 1.0 μm and most typically about 0.01 to 0.5 μm. The microparticle 10 may be made of any material compatible with the type of samples in which analytes will be detected. Example materials for any of the microparticles disclosed herein include, but are not limited to, polymers of styrenes, silicates, silica or carbon based aerogels or xerogels, silicones, latex, carbohydrates, methacrylates, acrylamides and the like, or any such materials that can be formed into a particle of the requisite size and be derivatized to contain the plurality of functional groups 20 on the surface of the microparticle 10. It is desirable but not necessary that the microparticle have a relative large void volume to material volume so that the microparticles in solution will have a density about the same as that of the sample so that the microparticles remain suspended in the sample without sinking.

The signal molecule 30 and the analyte binding molecule 40 are bound to the surface of the microparticle 10 in a ratio that typically, but not necessarily, provides more signal molecules 30 than analyte binding molecules 40. Typically, the ratio of signal molecules 30 to analyte binding molecules 40 is at least 3 to 1, at least 5 to 1, or preferably at least 10 to 1 or greater. The selection of the ratio of signal molecules 30 to analyte binding molecules 40 is ultimately determined by the affinity of the analyte binding molecule 40 for the analyte 50, relative to the signal amplification needed to provide detection within a desired sensitivity for a given sample type. Generally, as the affinity between the analyte 50 and the analyte binding molecule 40 increases, the less analyte binding molecule 40 is needed to provide reliable binding in a given sample, and therefore the higher the ratio of signal molecule 30 to analyte binding molecule 40 that can be used. The ratio of signal molecule 30 to analyte binding molecule 40 can be increased up to an empirically defined limit where the amount of analyte binding molecule 40 is too low to bind a sufficient number of analyte molecules 50 in a given sample in a given period of time to detect the analyte. One technique for empirically determining the ratio of signal molecules 30 to analyte binding molecules 40 is exemplified in Example 8.

The signal amplification microparticle 11 containing the signal molecule 30 and analyte binding molecule 40 serves as the principle vehicle for signal amplification in MBA according to the present invention. Signal amplification stems in-part from the fact that the microparticle 10 contains a large numbers of functional groups 20 coupled to a correspondingly large number of signal molecules 30 through the first attachment moiety 25. For example, one gram of 1-2 μm magnetic beads (Polysciences, Inc., Warrington, Pa.) contains 240 μmoles of amine groups on the particle surface, or about 3×10⁹ amine groups per particle. If only 0.01% of these amines were conjugated to signal molecules 30, each having only one acridinium signal emitting moiety 37, a common luminometer would still be able to reliably detect the presence of a single signal amplification microparticle 11. Accordingly, if only one analyte binding molecule 40 were attached to the signal amplification microparticle 11, that analyte binding molecule 40 would be associated with 3×10¹⁰ signal emitting moieties 36 also bound to the signal amplification microparticle 11. Thus, a first level of signal amplification is provided by the plurality of signal molecules 30 attached to the signal amplification microparticle 11. In contrast, an analyte binding molecule 40 alone can only be labeled with a few signal emitting moieties using conventional labeling techniques.

A second level of amplification stems from the fact that each signal molecule 30 attached to the signal amplification microparticle 11 is bound to a plurality of signal emitting moieties 36. The signal binding domain 34 on the signal molecule is preferably a polymer having at least 10, at least 20, at least 50 or at least 100 functional groups 33 to which the signaling moiety 36 is conjugated. Thus, the amplification of the signal by use of the signal amplification microparticle 11 bound to the plurality of signal molecules 30 is further multiplied by the plurality of signal emitting moieties 36 bound to each signal molecule 30, thereby providing at least 10 to 100 fold further amplification. For example, with a 0.5 μm signal amplification microparticle 11 having about fifty acridinium moieties 36 bound to each signal molecule 30, a conventional luminometer can reliably detect a single analyte molecule 50 bound to the microparticle 10 through a single analyte binding molecule 40, assuming only that the analyte bound signal amplification microparticle 11 can be obtained separately from non-analyte bound signal amplification microparticles 11 to remove background signals. As described in elsewhere herein, analyte bound signal amplification microparticles 11 can be removed from non-analyte bound signal amplification microparticles 11 by use of a second microparticle with an analyte binding molecule.

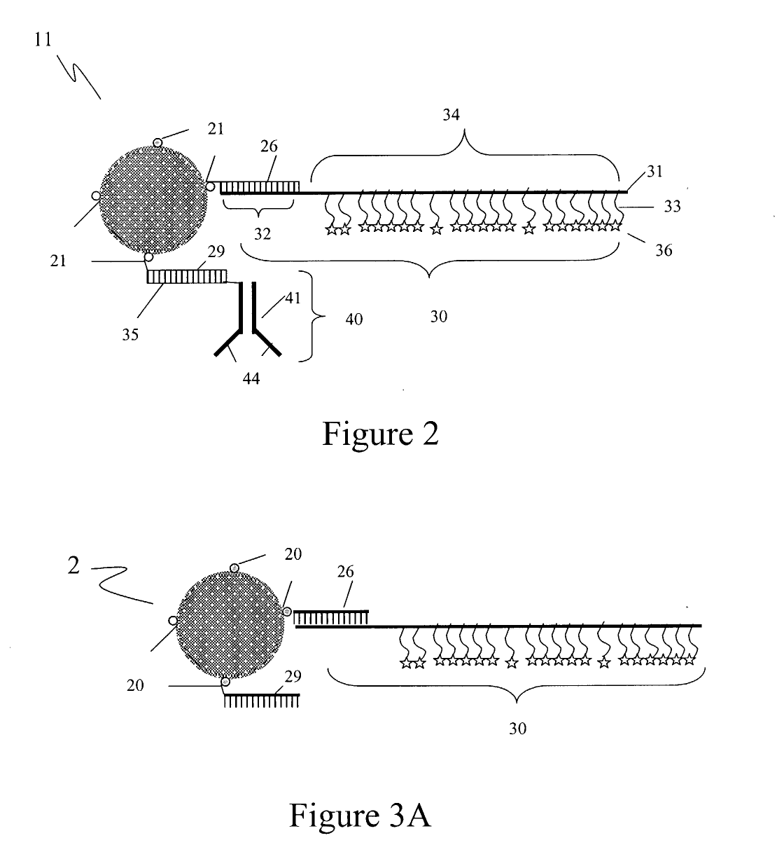

FIG. 2 illustrates an embodiment of the signal amplification microparticle 11 where the first and second attachment moieties, as well as the signal molecule, are comprised of oligonucleotides. The microparticle 10 is a 0.1 to 0.5 μm plastic bead derivatized to contain amine functional groups 21 on the surface. Such microparticles are commercially available from a variety of sources, for example, Bangs Laboratories (Fishers, Ind.) supplies microspheric beads of various sizes coated with various functional groups, including but not limited to amino, carboxyl, hydroxyl, sulfhydryl, hydrazide, amide, chloromethyl, aldehyde, epoxy and tosyl groups and also supplies techniques for covalently coupling the same as described in TechNote # 205, which is incorporated herein by reference. The first attachment moiety 26 is a first oligonucleotide of suitable length, for example 30 bases, with a defined nucleic acid sequence. The second attachment moiety 29 is also an oligonucleotide (i.e., a second oligonucleotide) of about 30 bases in length with a defined nucleic acid sequence. It will be understood by one of ordinary skill in the art that the length and sequence of the first 26 and second 29 attachment oligonucleotide moieties can be of any composition to provides a stable and rapid binding by base pairing with a complementary oligonucleotide. Typically, the length and sequence should be selected to provide a melting temperature of at least about 50° C., and more typically, at least about 70° C. The 5′ ends of the first and second oligonucleotides 26 and 29 are cross linked to the amine functional groups 21 on the surface of the microparticle 10 by a cross linking reagent. One example cross linking reagent suitable for this purposes is BS³ [Bis(sulfosuccinimidyl)suberate], a homobifunctional NHS-ester.

The signal molecule 30 is an oligonucleotide 31 (i.e., a third oligonucleotide) of at least 50, at least 100, or at least 200 nucleotides in length. In a typical embodiment, the third oligonucleotide 31 is about 250 nucleotides in length and includes two domains. The first domain is the conjugation domain 32, which is located at the 5′ or 3′ end of the third oligonucleotide 31. The conjugation domain 32 includes a nucleotide sequence complementary to the first oligonucleotide attachment moiety 26. The second domain is the signal binding domain 34, which includes the remainder of the nucleotide residues in the third oligonucleotide, each of which contain the plurality of functional groups 33. One example of a suitable cross linking group 33 is a primary amine, a commonly used functional group that can be introduced during oligonucleotide synthesis. The plurality of linker residues 33 of the third oligonucleotide 33 are cross linked to a plurality of acridinium ester molecules 37 to form the signal emitting moieties 36 of the signal binding domain 34 of the third oligonucleotide 33. Suitable methods for incorporating primary amines into oligonucleotides during oligonucleotide synthesis include, but are not limited to, the use of nucleotide phosphoamidites modified to contain a primary amine, or the use of non-nucleotide phosphoramidite designed for incorporating primary amines into oligonucleotides. These phosphoramidites can be efficiently incorporated into oligonucleotides and are readily available from commercial sources, e.g., Glen Research (Sterling, Va.) for primary amine derivatized dT or dC phosphoramidite and Clontech (Palo Alto, Calif.) for primary amine derivatized non-nucleotide phosphoramidite called UniLink™ AminoModifier. The primary amine groups in the phosphoramidites are normally protected during oligonucleotide synthesis and deprotected after the completion of oligonucleotide systhesis thereby freeing the primary amines. After preparation of the signal molecule 31 with attached acridinium signal emitting moieties 37, the signal molecule 31 is conjugated to the first oligonucleotide attachment moiety 26 by hybridization of the nucleotides in the conjugation domain 32 to the complementary nucleotides of oligonucleotide attachment moiety 26.

The analyte binding molecule 40 includes the second conjugation domain 35, which is an oligonucleotide complementary to the second oligonucleotide attachment moiety 29. The second oligonucleotide conjugation domain 35 is conjugated to the analyte binding molecule 40 using conventional cross-linking chemistry. Cross linking of the second oligonucleotide conjugation domain 35 to the analyte binding molecule may utilize, for example, functional groups that are normally, but not necessarily, derivatized onto the 3′ or the 5′ nucleotides of oligonucleotide conjugation domain 35. These functional groups include, but are not limited to, primary amines, thio groups and carboxylic acids which are cross linked to functional groups of the analyte binding molecule 40 such as amines, thio groups, carbonyls, carboxylic acids and the like, which are typically present on a protein type analyte binding molecules such as antibodies, receptors and/or other polypeptide containing species.

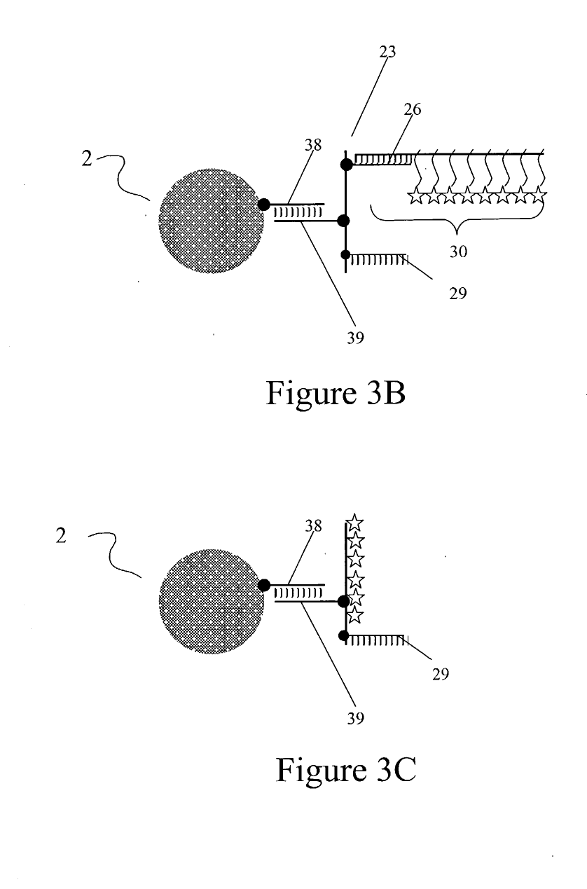

The core microparticle composition 2 depicted in FIG. 1 provides a convenient platform for assembling a variety of signal amplification microparticles 11 with any desired specificity. As depicted in FIG. 3A, each of the first and second attachment moieties 26 and 29 are oligonucleotides attachment moieties. The core microparticle composition 2 can be made with a combination of defined oligonucleotide attachment moieties 26 and 29. Such a core microparticle composition 2 is convenient to make because a single batch of microparticles 10 having the same first oligonucleotide attachment moiety 26 and the same second oligonucleotide attachment moiety 29 can be made at one stage, where oligonucleotides 26 and 29 are cross linked to the functional groups 20 on the surface of microparticle 10 in the same reaction. The molar ratio of the oligonucleotides 26 and 29 used in the reaction determines the ratio of the oligonucleotides 26 and 29 linked to the microparticle 10. The core microparticle composition 2 can subsequently be combined with different batches of the third oligonucleotide 31 signal molecule and/or with different batches of analyte binding molecules 40 at another stage. The sequences of the first 26 and second 29 oligonucleotides may be the same or may differ in various embodiments, however, where a core signal amplification microparticle 2 is desired to serve many detection methods, the first oligonucleotide attachment moiety 26 typically has a sequence that differs from the second oligonucleotide attachment moiety 29. In this manner, a reproducible set of hybridizing conditions can be defined to optimally conjugate the oligonucleotide signal molecule 31 and the analyte binding molecule 40 in separately controlled steps. Thus, a non-specific signal amplification microparticle 11 can first be made using the core microparticle 2 conjugated to a common batch of oligonucleotide signal molecules 31 and then subsequently made into a specific signal amplification microparticle 11 in a separate step.

FIGS. 3B and 3C depict two alternative methods of indirect conjugation of the first 26 and second 29 oligonucleotides to microparticles to form the core signal amplification microparticle 2. In both methods, a vehicle molecule 23 (e.g., polylysine) with a plurality of functional groups serves as a vehicle, to which multiple oligonucleotides are covalently conjugated. In certain embodiments, as illustrated in FIG. 3B, at least three distinct oligonucleotides are linked to vehicle molecule 23. One of the oligonucleotides 39 is complementary to oligonucleotide 38, which is covalently conjugated to the microparticle. The first 26 and second 29 oligonucleotides are attached to the vehicle molecule 23 as well. In other embodiments, as depicted in FIG. 3C, where the vehicle molecule 23 contains sufficient numbers of functional groups to permit direct coupling of sufficient numbers of signal emitting molecule 37 (e.g. acridinium), the presence of the first oligonucleotide 26, and subsequent attachment of signal molecule 30, becomes unnecessary. In these embodiments, the second oligonucleotide 29 is attached to the vehicle molecule 23.

In certain embodiments, as depicted in FIGS. 3D to 3G, the signal emitting molecule 37 (e.g., acridinium) is replaced with a signal generating molecule 43 (e.g., alkaline phosphatase), which itself does not emit signal but rather catalyzes the formation of a signal (e.g., light or color) from an appropriate substrate. In parallel to the attachment methods for signal emitting molecules 37, the attachment of signal generating molecule 43 to the signal amplification microparticle can also be accomplished through similar means. The signal generating molecule 43 can be conjugated to a vehicle molecule 23 directly (FIG. 3E) or indirectly through oligonucleotide hybridization (FIG. 3D). Alternatively, the signal generating molecule 43 can be coupled to the signal amplification microparticle directly (FIG. 3G) or indirectly through oligonucleotide hybridization (FIG. 3F).

To provide analyte specificity to the signal amplification microparticle 11, all that is needed is to conjugate the specific analyte binding molecule 40 by hybridization to the second oligonucleotide conjugation domain 35. In the embodiment depicted in FIG. 2, where the analyte binding molecule is an antibody 41 cross linked to the oligonucleotide conjugation domain 35. The analyte binding domain 44 of the antibody 41 is the ordinary antigen binding domain that includes the variable region(s) of the antibody molecule. Because the antibody 41 provides the analyte specificity, one batch of the core microparticles 2 assembled with the signal molecules 30 can be used with a variety of different antibodies to make a variety of specific signal amplification microparticles 11, reducing the amount of process control monitoring required to assure production of acceptable signal amplification microparticles 11. It will be understood that the term “antibody” as used herein, includes any antigen recognizing polypeptide obtained from an animal, or which otherwise contains amino acid sequences having variable region binding domains encoded by DNA that encodes various components of the immune system of an animal. This includes, but is not limited to polyclonal antibodies, monoclonal antibodies, antibody phage display sequences, any class of immunoglobulins, or T-cell receptors. The term also includes functional antigen binding fragments thereof, including, for example, Fab or Fab1′ or chimeric fusion molecules containing the variable region of any antigen binding domain.

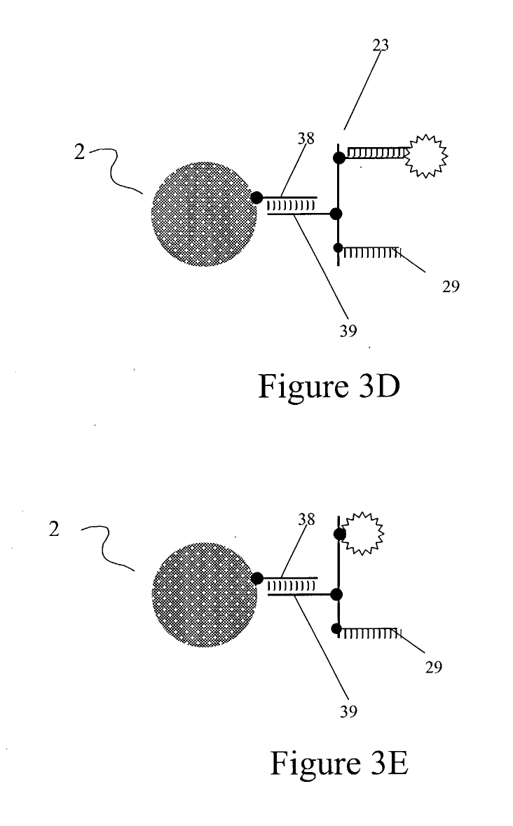

The signal amplification microparticle 11 is used in a system for detecting specific analytes. An embodiment of an analyte detection system 51 using the signal amplification microparticle 11 is depicted in FIG. 4. In addition to the first microparticle 10 assembled to form the signal amplification microparticle 11, the analyte detection system includes a second microparticle 60 assembled to form separating microparticle 73. The second microparticle 60 has a different physical structure from the first microparticle 10, which allows ready separation of the signal amplification microparticle 11 from the separating microparticle 73 unless the two particles are associated in a complex 65 by cross binding to the analyte 50, in which case the complex 65 is also separated from non-complexed signal amplification microparticles 11. Example differences in physical structure that permit separation from non-complexed signal amplification microparticles 11 include, but are not limited to, difference in size, density, electrical, magnetic or florescent properties. In one example embodiment, the separating microparticle 60 is larger than the signal amplification microparticle 11. In another embodiment the separating microparticle 60 includes ferromagnetic components 62 so that the separating microparticle 60 constitutes a ferromagnetic bead that can be separated from a non-ferromagnetic signal amplification microparticle 11 using a magnet. In another embodiment the separating microparticle 60 is both ferromagnetic and larger than the signal amplification microparticle 11. In other embodiments, the second microparticle 60 may be tagged with a florescent label to permit it to be selectively removed from a sample using a florescence activated cell sorter (FACS) configured to sort particles based on fluorescence. In still other embodiments, the separating component is an immobile solid phase such as microtiter plate wells rather than separating particles.

In typical embodiments, the second microparticle 60 has a diameter of at least about 1 ?m, typically about 1 to about 10 ?m, and most typically about 1 to about 5 ?m. It is understood that any of the microparticles referred to herein, such as microparticle 10 and/or microparticle 60 are not necessarily spherical in shape although they may be denominated nanospheres or microspheres. Microparticles 10 or 60 may in fact be of irregular in shape. The term “diameter” is therefore understood to refer to the average length of the largest dimension in a population of microparticles. When the separating microparticle 73 is comprised of a ferromagnetic second particle 60 the separating microparticle 73 can be isolated from a mixture or retained against a wall of container by use of a magnet while the signal amplification microparticle 11 remains free in suspension and can therefore be washed away from the separating microparticle 73 unless the signal amplification microparticle 11 is associated with the separating microparticle 73 in the complex 65.

The separating microparticle 73 cross binds to the analyte 50 through a second analyte binding molecule 70. The second microparticle 60 like the first microparticle 10 has a plurality of functional groups 61 (i.e., third functional groups) on its surface. These functional groups 61 may be conjugated directly to the second analyte binding molecule 70 as illustrated in FIG. 4A or indirectly thereto, through a third attachment moiety 65 as illustrated in FIG. 4B. In certain embodiments, the third attachment moiety 65 is a third oligonucleotide attachment moiety 66 analogous to the first and second oligonucleotide attachment moieties 26 and 29 of the signal amplification microparticle 11. The second analyte binding molecule 70 has a conjugation domain 71 (i.e., a third conjugation domain) and a second analyte binding domain 74 analogous to the second conjugation domain 35 and fist analyte binding domain 44 of the first analyte binding molecule 40 attached to the signal amplification microparticle 11. The second analyte binding molecule 70 on the separating microparticle 73 may be the same type of molecule as the first analyte binding molecule 40, or may be a different type. For example, in one embodiment, the first 40 and second 70 analyte binding molecules are polyclonal antibodies obtained from an animal immunized with an antigen analyte 50. In other embodiments, the first 40 and second 70 analyte binding molecules may be monoclonal antibodies expressed in different hybridoma cell lines.

It is preferred that the first 40 and second 70 analyte binding molecules bind to a different structural feature on the analyte 50 to facilitate association of a plurality of the signal amplification microparticles 11 with a plurality the separating microparticles 73 in the formation of the complex 65. For the sake of clarity, FIG. 4A illustrates the complex 65 having only one signal amplification microparticle 11 associated with only one separating microparticle 73, however, it is understood that in practice, there will be a large number of signal amplification microparticles 11 associated with a large number separating microparticles 73 so that the complex 65 actually forms into an aggregated lattice. The complex 65 includes at least one analyte molecule 50, at least one signal amplification microparticle 11 and at least one separating microparticle 73. The formation of the complex 65 is akin to an agglutinating reaction between antibodies and antigens or between antibodies bound to beads through an antigen. When the analyte 50 is a large species such as a bacterium, virus or a macromolecule, multiple separating microparticles 73 and multiple signal amplification microparticles 11 may bind to a single analyte 50. In such cases, formation of the complex 65 still further amplifies the detection sensitivity because multiple signal amplification microparticles 11 are then associated with a single analyte in forming the complex 65. The complex 65 is readily separated from non-complexed signal amplification microparticles 11 by virtue of the separation property of the separating microparticle 73 thereby greatly reducing background noise from non-complexed signal amplification microparticles 11.

The complex 65 typically sediments more rapidly than the signal amplification microparticle 11 allowing the complex 65 to be readily separated from the suspension by techniques such as centrifugation in certain embodiments. Formation of the complex 65 also creates a larger structure than either the signal amplification microparticle 11 or separating microparticle 73 alone, allowing separation of the complex 65 from the signal amplification microparticle 11 by filtration in other embodiments. The aggregation complex 65 may also be adhered to the surface of a microtiter plate in still other embodiments by adhering the separating microparticle 73 to the surface of the plate. Use of centrifugation, filtration or adherence, while useful in some applications, adds additional steps relative to the preferred methods provided herein, which are designed to use MBA for rapid and economical detection of small quantities of analytes.

It is therefore preferred that the complex 65 be separated from non-complexed signal amplification microparticles 11 by less cumbersome techniques. As mentioned above, use of a ferromagnetic separating microparticle 73 (i.e., a magnetic bead) permits washing away of signal amplification microparticles 11 not associated with the separating microparticle 60. After incubation of the signaling particle 11 and the separation particle 73 in the presence of an analyte, a magnet is used to attract the separation particle 73 to a specific location, for example to the side of a test tube, so that all the signaling particles, except those not linked to the separation particle 73 can be removed from a reaction mix while retaining the bulk of the separation particles. Removal of the magnet from proximity to the test tubes release the separation particles, which can then be washed with an appropriate wash solution, then re-attracted to the magnet so that the wash effluent is removed. After one to three washes, the separating microparticles 73 will be retained and some of these will be associated with the signal amplification microparticles 11 via cross binding to the analyte by the first and second analyte binding domains 44 and 74 of the first 40 and second 70 analyte binding molecules in the complex 65. The amount of associated signal amplification microparticles 11 in the retained complex 65 will directly correspond to the amount of analyte originally present.

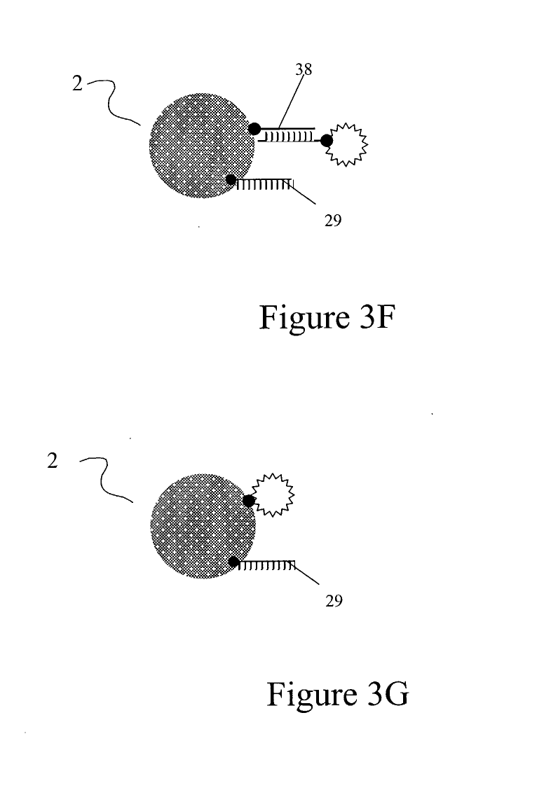

Accordingly, another aspect of the invention includes methods of detecting an analyte based on use of the signal amplification microparticle 11 in combination with the separating microparticle 73. FIG. 5 generally illustrates one embodiment of such a method. The method includes contacting a sample containing the analyte 50 with the signal amplification microparticle 11 containing the first analyte binding molecule 40 and signal molecule 30. The sample is also contacted with the separating microparticle containing the second analyte binding molecule 70 to form a mixture. The sample contact with separating microparticle 73 and signal amplification microparticle 11 may be performed in one step, as generalized in FIG. 5A, or in two steps as summarized in FIG. 5B. The mixture is incubated for time sufficient for the first analyte binding molecule 40 and the second analyte binding molecule 70 to bind the analyte and to form the complex 65. Typically the mixture is incubated for less than 20 minutes, less than 10 minutes or less than five minutes. The signal amplification microparticles 11 not bound to the complex 65 are then removed by separating the complex 65 from non-bound signal amplification microparticles 11 using the differential physical property of the separating microparticles 73 to form a retained fraction containing the complex 65. Optionally, and preferably, the retained fraction is washed at least once to further remove non-bound signal amplification microparticles 11 that may be non-specifically associated with the complex 65. The retained complex is then placed under conditions that cause the signal to be emitted from the signaling moieties 36 on the signal amplification microparticles 11 retained in the complex 65. The amount of signal emitted is measured, which corresponds to the amount of analyte in the complex, which in turn corresponds to the amount of analyte in the sample. In preferred embodiments, the entire method is executed in less than 30 minutes, less than 20, minutes or less than 15 minutes.

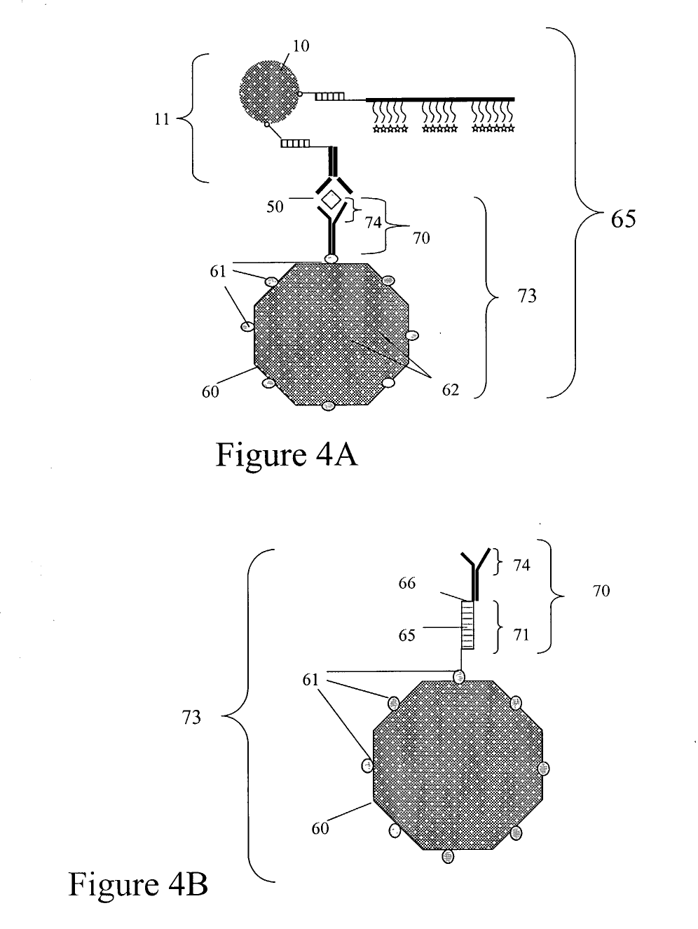

FIG. 6 illustrates an embodiment of a kit for executing a method for detecting the presence of bacteria in a sample. The analyte 50 in this embodiment is the bacterium 53, which has a plurality of antigenic sites 54 on its surface. The signal amplification microparticle 11 includes an oligonucleotide signal molecule 31 labeled with a plurality of acridinium moieties 37 linked to the nucleotides in the signal binding domain 34 of the signal molecule 31. The signal molecule 31 is conjugated to the first oligonucleotide binding moiety 26 on the surface of the first microparticle 10 by complementary base pairing between the first oligonucleotide binding moiety 26 and the first conjugation domain 32. A polyclonal antibody 41 specific for a surface antigen 54 on the bacterium 53 is conjugated to the first microparticle 10 by base pairing between the second oligonucleotide attachment moiety 29 and a complementary oligonucleotide linked to the antibody 41, which comprises the second conjugation domain 35. The antibody 41 in turn binds the antigen 54 on the surface of the bacterium 53 at a first binding site. The separating particle 73 includes the polyclonal antibody 41 bound to a ferromagnetic separating microparticle 60. In embodiments such as depicted in FIG. 4B, the polyclonal antibody 41 is the second analyte binding molecule 70 and which is conjugated to a third oligonucleotide 71 which forms the third conjugation domain, which is in-turn conjugated to the microparticle 60 through base pairing between the third oligonucleotide attachment moiety 66 and the third oligonucleotide 71. After mixing the signal amplification microparticle 11 with the sample containing the bacterium 53 and the separating microparticle 73, the complex 65 is separated by magnetic attraction and washed as described above. The amount of light emitted from the acridinium moieties in the washed complex 78 is measured and compared to a standard curve to determine the number of bacterium 53 present in the sample or compared to the amounts of light emitted in a negative control in a qualitative assay.

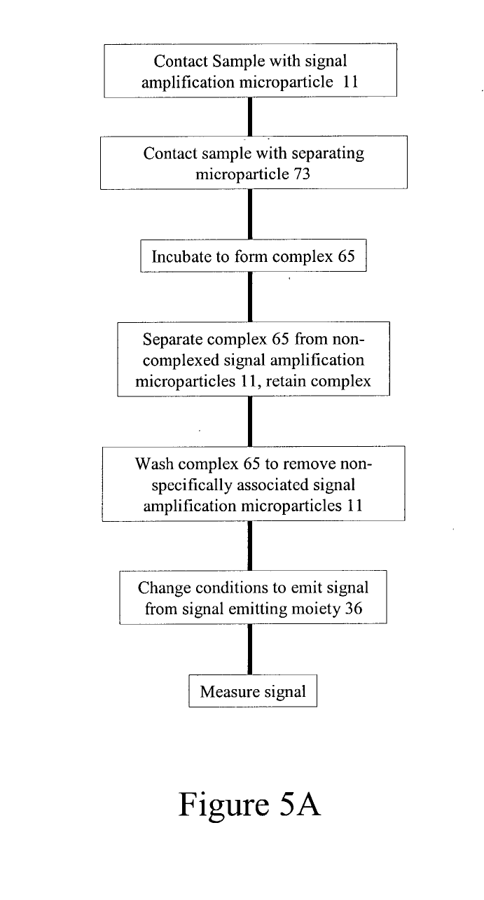

FIG. 7 illustrates an embodiment of a kit for detecting an antibody analyte 79, where the analyte binding molecules are antigens 81, 82. The antigens 81 and 82 are conjugated to the surface of the signal amplification microparticle 11 and the separating microparticle 73, respectively. In one embodiment, antigens 81 and 82 are human immunodeficient virus (HIV) antigens, such as an envelope protein of an HIV particle or viral proteins isolated from a viral lysate. One or both of the antigens 81 and 82 may be conjugated to the microparticles through an attached oligonucleotide conjugation domain such as the conjugation domain 35 that binds to the oligonucleotide attachment moiety 29 as illustrated by the signal amplification microparticle 11 in FIG. 7. Alternatively, one or both of the antigens 81 and 82 may be directly cross linked to at least one of microparticles as indicated by the separating microparticle 73 in FIG. 7. The target antibody 79 is detected in the sample by binding of the antibody 79 to the antigens 81 and 82 on both the signal amplification microparticle 11 and the separating microparticle 73. When the separating microparticle 73 is separated and washed as described herein before, the amount of signal amplification microparticle 11 retained with the separating microparticle 75 is compared to standard curve to determine the amount of the antibody 83 in the sample.

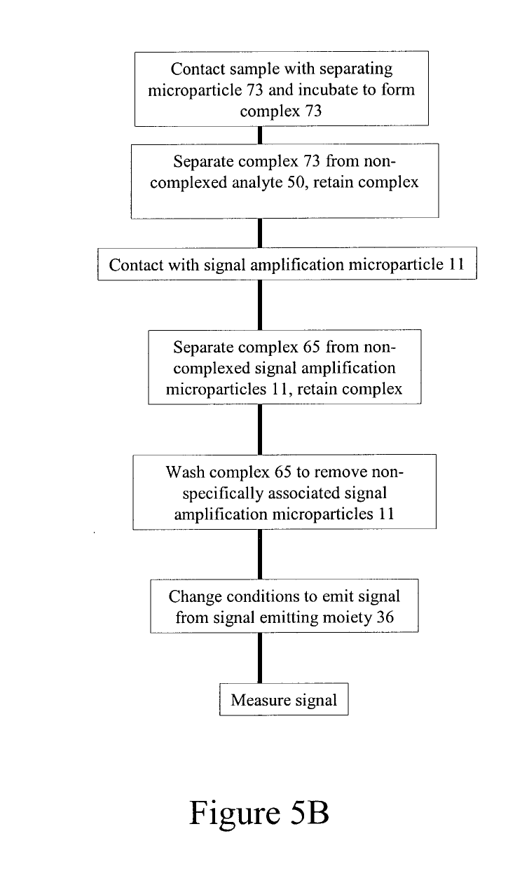

While FIG. 7 illustrates an embodiment where the antigens 81 and 82 are macromolecular structures such as HIV envelop proteins, one of ordinary skill in the art will appreciate, that any antigenic determinant can be incorporated into the signal amplification microparticle 11 and separating microparticle 73 using conventional technology. For example, as depicted in FIG. 8, where one or both of antigens 81 and 82 are small molecule epitopes, for example peptides or other haptens, the same can be readily linked to a suitable carrier molecule 84 such as keyhole limpet anthocyanicn, E. coli toxin, ovalbumin and the like. In such cases, the carrier molecule 84 becomes a component of the analyte binding molecules 40 and/or 70 and the carrier molecule 84 contains the attached conjugation domains 35 and or 71 at one region of the carrier molecule 84 and contains the cross linked antigen 81 at another region. The antigens 81 and 82 used on the signal amplification microparticle 11 and separating microparticle 73 may have the same structure or have different structures.

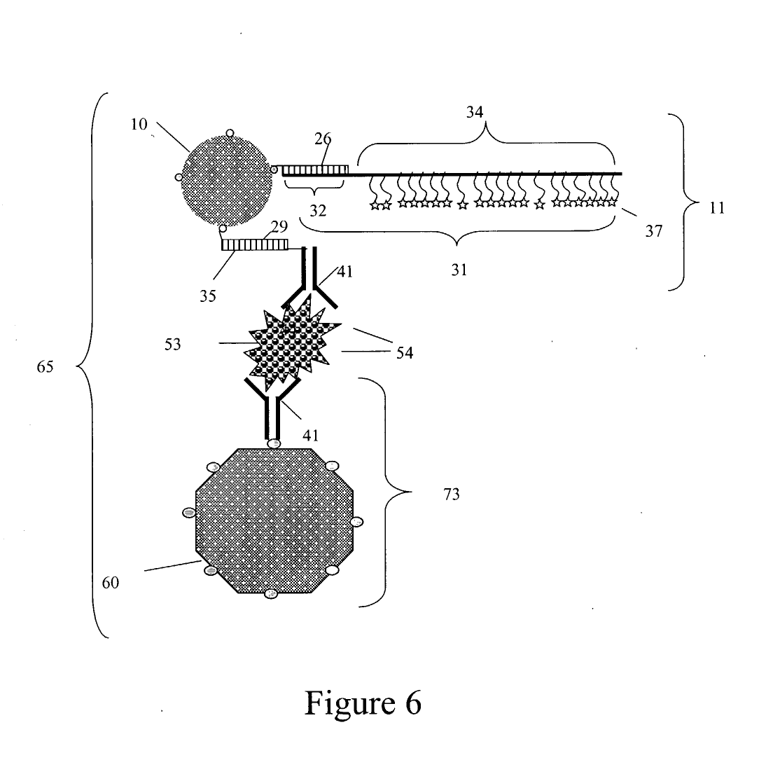

Various embodiments may utilize combinations of different types of analyte binding molecules. For example, with reference to FIG. 9, the signal amplification microparticle 11 is conjugated to the antigen 81 that includes the epitope recognized by the target antibody 79 being detected. The separating microparticle 73 on the other hand, is conjugated to a generic anti-species antibody 42, for example, rabbit anti-human IgG that binds to all human IgG molecules. In this example, the separating microparticle 73 will non-specifically bind to all human antibodies 79 through analyte binding antibody 42, but the only signal that will be detected will be determined by the amount of the antibodies 79 that also specifically bind the antigen 81 analyte binding molecule present on the signal amplification microparticle 11.

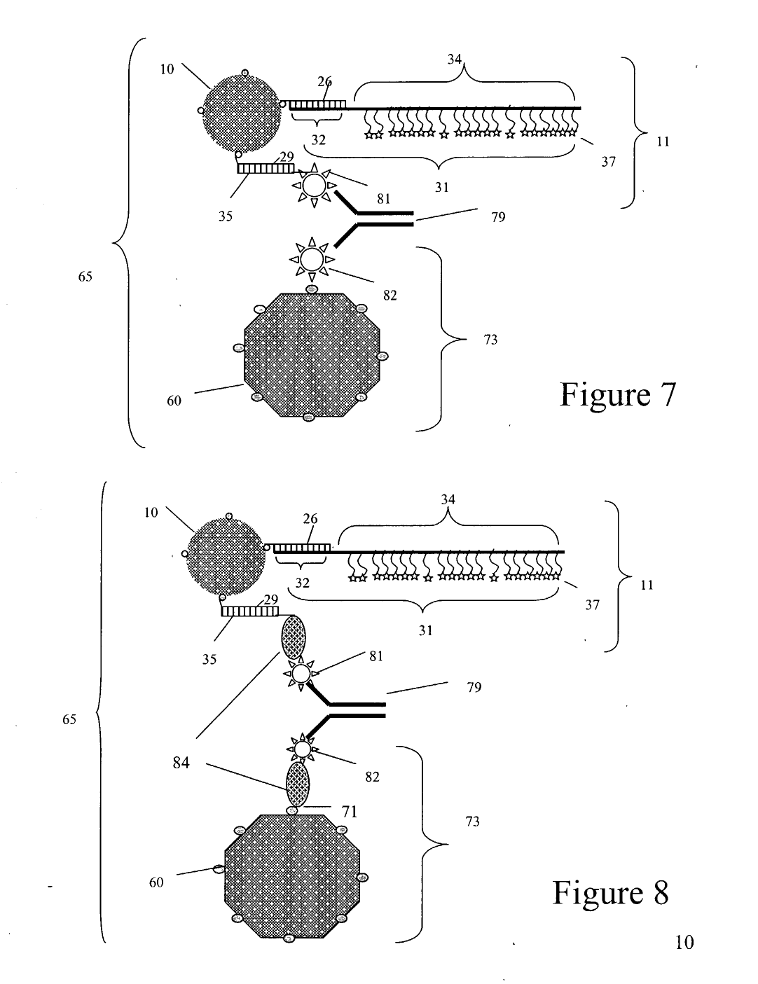

FIG. 10 illustrates an embodiment for detecting a nucleic acid sequence as the analyte 90. In this embodiment the first analyte binding molecule (40) is a fist nucleic acid sequence 89 where the conjugation domain 91 includes sequence complementary to the second oligonucleotide acid attachment moiety 29 and the analyte binding domain 92 comprises a nucleic acid sequence complementary to a first target sequence 94 on the nucleic acid analyte 90. The first nucleic acid sequence 89 is bound to the signal amplification microparticle 11 through the conjugation domain 29 and binds the target nucleic acid analyte 90 at the target sequence 94 through complementary base pairing with the analyte binding domain 92. The separating microparticle 73 includes a second nucleic acid sequence 95 that has a conjugation domain sequence 71 complementary to the (third) oligonucleotide acid attachment moiety 66 on the separating microparticle 73. The second nucleic acid sequence 95 also includes a second analyte binding domain 97 that is a nucleic acid sequence complementary to a second target sequence 98 on the target nucleic acid analyte 90. When mixed with a sample containing the nucleic acid analyte 90 under conditions that promote hybridization, the first and second target nucleic sequences 94 and 98 of the target nucleic acid 90 bind the signal amplification microparticle 11 and the separating microparticle 73 to form the complex 65. The amount of signal in the complex 65 is compared to standard curve to determine the amount of the target nucleic acid analyte 90 present in the sample. While FIG. 10 depicts one binding site for either signal amplification microparticle 11 or separating microparticle 73, it is understood that more than one binding sites on the analyte molecules can be targeted and are preferred. It is also understood that analyte binding molecules 89 and/or 95 may not necessarily be fist nucleic acid sequences. They may only contain analyte binding domains and, in this case, are directly conjugated to the microparticle 10 and/or the separating microparticle 60.

In the embodiments described above, the various attachment moieties 25, 28 and 65 have been depicted primarily as oligonucleotides 26, 29 and 66, respectively. The versatility of using oligonucleotide attachment moieties has been mentioned hereinbefore. The invention is not, however, limited to using oligonucleotide attachment moieties. As mentioned above, in certain embodiments, the signal molecule 30 and/or the analyte binding molecules 40 and 70 can be directly attached to their respective microparticle 10 and/or 60 by direct conjugation to functional groups 20 and 61, or through conjugation to an intervening chemical moiety (such as the carrier molecule 84) attached to the microparticles 10 or 60.

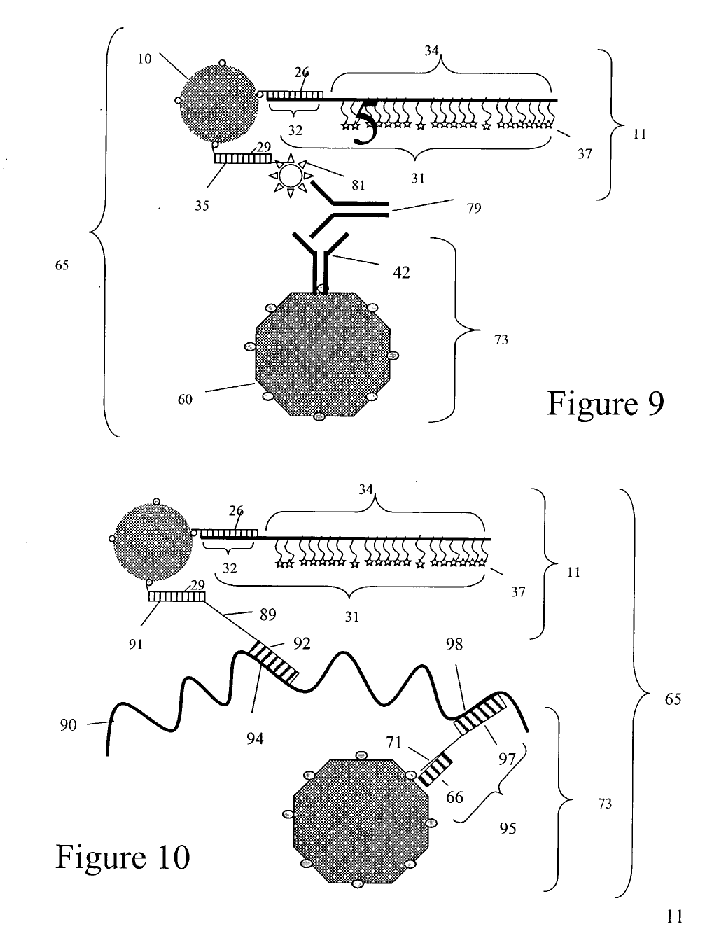

FIG. 11 illustrates an embodiment where direct chemical cross-linking is used to attach the signal molecule 30 and the analyte binding molecules 40 and 70 to the signal amplification microparticle 11 and the separating microparticle 73. In such embodiments, the attachment moieties 25, 28 and/or 65 may be defined by the appropriate chemical terms for the R group substitutent of the bond linkage at the junction between the functional groups 20 or 61 on the microparticles 10 and 60 and the residues 32, 35 and/or 72 to which the microparticles 10 and/or 60 are cross-linked. In such cases, the binding domain 32 of the signal molecule 30 and/or the binding domains 35 or 72 of the analyte binding molecules 40 and 70 are defined by the corresponding chemical term for the residues on those molecules that form part of the bond. Thus, the bond linkages defined by functional groups 20 and 61, the R groups 25, 28 and 65 and the residues 32, 35 and 72 also define their respective attachment moieties and binding domains.

### EXAMPLE 1

### Bacterial Assay Using Microparticle Based Amplification

Bacterial preparation: A laboratory strain of E. coli, M15 that carries a kanamycin resistance gene is grown on a LB plate and used to inoculate 4-mL of adult bovine serum containing 25 μg kanamycin/mL. The cells are allowed to propagate for 20-24 hours at 30° C. with shaking (250 rpm). Two mL of the culture is used to inoculate 300 mL of bovine serum containing kanamycin. The cells are grown overnight (about 15 hours) at 30° C. with shaking (250 rpm). About 40 mL of the culture is removed, 10 mL glycerol is added and the mixture is aliquoted into 1 mL portions, to be used for preparation of standards and positive controls. A bacterial titer is taken by plating 20 μL of a 1:500 and a 1:1000 diluted sample onto an agar medium supplemented with 25 μg Kan/mL followed by overnight growth at 37° C. and counting of the colonies and calculate the bacterial concentration in the glycerol stock.

The remaining cells from the 300-mL are recovered by centrifugation at 6000×g for 5 minutes and suspended in 10-mL PBS. The suspended cells are diluted to 250-mL PBS and recovered by centrifugation and washed by suspension in 250-mL PBS followed again by centrifugation and are finally suspended in 10 mL PBS. The cell suspension is then inactivated by incubation at 90° C. with intermittent shaking for 90 minutes. Fifty microliters of the incubated suspension is plated on Kan-LB plate medium to ensure that there are no live bacteria remaining.

Polyclonal antibody production. Polyclonal antibodies to the inactivated bacterial cells are obtained by immunization of rabbits or other animals by any of a variety of methods known to those of skill in the art. As one resource, a commercial enterprise such as ProSci Inc., (Poway, Calif.) that specializes in antibody production may be used to obtain antibodies on a commercial scale. The immunization protocols typically used with inactivated bacteria typically include immunizing rabbits with about 10⁸ bacterial cells using incomplete Freund's Adjuvant because complete Freund's adjuvant contains bacterial components. The rabbits are boosted twice at two-week intervals and antisera are recovered by bleeding the rabbits and recovery of the sera from separated blood cells.

Affinity purification of antibodies. Bacteria are used as an affinity matrix for affinity purification of polyclonal antibodies. Bacteria are grown to late log phase in 1.0 Liter of bovine serum medium containing 3 grams of glucose. The cells are collected by centrifugation at 6000×g for 5 minutes and then suspended in 250-mL of PBS. After centrifugation to collect cells, the cell pellet is suspended in 250-mL of elution buffer (0.5 M acetate, 0.15 M NaCl, pH 2.4). The cells are pelleted and washed with 250-mL of PBS. The bacterial cells are then completely suspended in 10 mL PBS.

The antiserum (20-40 mL) is first adjusted to 1×PBS using 10×PBS, diluted with 1×PBS to about 1-2 mg protein/mL and then mixed with the bacterial cells. Incubate at room temperature for 30-40 minutes with mild agitation. The mix is diluted to 250 mL with PBS. The cells are pelleted, washed twice with 250 mL of PBS, and then suspended in 30-mL of elution buffer. After incubation at room temperature for 10 minutes, the cells are pelleted. Collect the supernate. The cells are suspended again in 20 mL of elution buffer, incubated at room temperature for 10 minutes and pelleted. Collect the supernate. Combine the supernate and dialyzed at 2-8° C. overnight against 2-4 L of PBS with 0.1% Tween 20. The solution is subjected to centrifugation at 12,000×g for 30 minutes and then concentration using ultrafiltration to 2-5 mg protein/mL. The IgG fraction of the purified antibodies may be further purified using a protein A column or a goat-anti-rabbit IgG column.

Determination of antibody titer. A common quantitative method for determining the titer of an antibody preparation is preferred for the purpose of quality control and reproducibility of an assay that may use similar procedures but use different samples to obtain the antibody preparation. Here, titer is defined as the antibody concentration (or dilution) that gives a detectable signal over the background in the following assay: The following represents one example protocol:

Two thousand (2000) heat-inactivated bacterium are spiked into each of six 1.0-mL volume of binding buffer (PBS with 10% bovine serum). Two 1.0-mL volumes of binding buffer without bacterium are used as a negative control. To the tubes containing bacteria, add the following amounts of antibodies respectively: 10 μL (1:100 final dilution), 5 μL (1:200 final dilution), 2.5 μL (1:400), 12.5 μL of 1:10 diluted (1:800 final dilution), 6.25 μL of 1:10 diluted (1:1600 final dilution) and 3.12 μL of 1:10 diluted (1:3200 final dilution). Incubate at room temperature for 20 minutes with gentle agitation. Wash four times with PBS by centrifugation (6,000 rpm for 5 minutes in a microfuge) and suspension. Finally the bacteria (and the controls) are suspended in 1.0-mL of binding buffer.

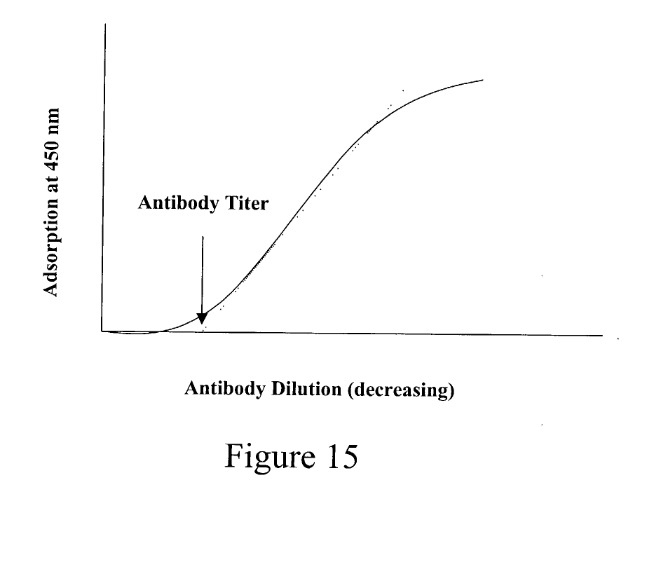

Add 5 μL (or as directed by the manufacturer) of goat anti-rabbit antibody conjugated with peroxidase. Incubate at room temperature for 25 minutes with gentle agitation. Wash four times with PBS by centrifugation (6,000 rpm for 5 minutes in a microfuge) and suspension. The bound peroxidase activity is measured by TMB method. Briefly, 10-mg TMB (Sigma, T2885) is dissolved in 1 mL DMSO, which is diluted into 99 mL 0.1 M sodium acetate buffer, pH 6.0. Just prior to use, 33 μL of hydrogen peroxide (30% W/W) is added to the solution. One hundred and fifty (150) μL of the resulted solution is then added to each well followed by incubation in the dark for 15-30 minutes. Add 50 μL of 2-M sulfuric acid to stop the reaction. Measure the adsorption at 450 nm using a spectrophotometer using the negative control as the background. Plot the data. The titer is the interception point between the linear line and the X-axis representing antibody dilution, shown in the FIG. 15.

### EXAMPLE 2

### Oligonucleotides: Sequence, Synthesis and Ligation

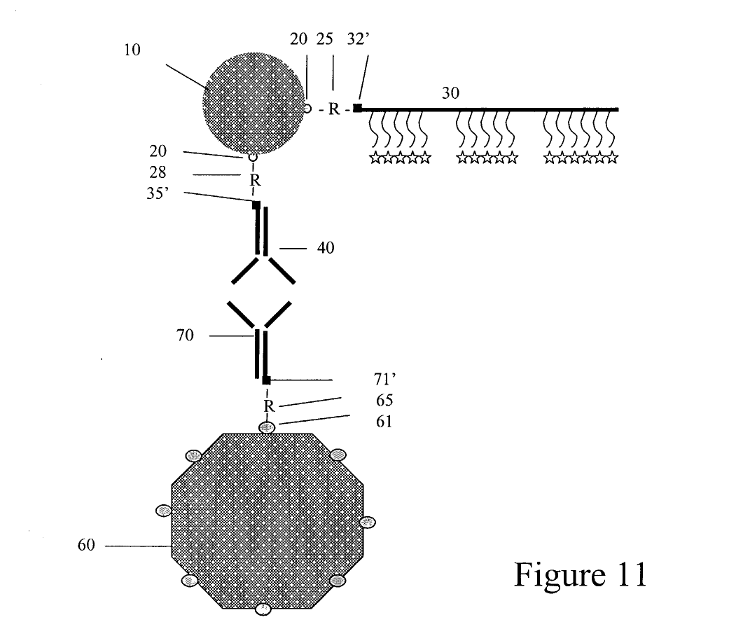

Design of oligonucleotides: FIG. 12 depicts four oligonucleotides, A-D. Their sequences are based on randomly generated sequences for the attachment oligonucleotide moieties 26 and 29. Oligonucleotide A represents the first oligonucleotide attachment moiety 26 which, in this example, is comprised of dA₍₄₀₎ i.e., 40 deoxy adenosine residues. Oligonucleotide A is complementary to the 3′ end of oligonucleotide C that forms the conjugation domain 32 of the signaling molecule 30, the 3′ end being comprised of 40 deoxy thymidine residues. Oligonucleotide B represents the second oligonucleotide attachment moiety 29, which in this example, is comprised of dG₍₃₀₎ i.e., 30 deoxy guanosine residues. Oligonucleotide B is complementary to oligonucleotide D which is comprised of dC₍₃₀₎ i.e., 30 deoxy cytidine residues which form the conjugation domain 35 that will be linked to the antibody 41 that serves as the analyte binding molecule 40. The complementary regions for each oligonucleotide pair have a melting temperature above 70° C. All oligonucleotides are synthesized with a conventional oligonucleotide synthesizer.

Oligonucleotide C is 190 bases long and in addition to the 40 residues at the 3′ end comprised of deoxy thymidine, further includes 150 residues for the signal binding domain 34. When oligonucleotide synthesis technology permits highly efficient synthesis of long oligonucleotides, the entire oligonucleotide C sequence of 190 bases may be synthesized as a single oligonucleotide. Otherwise, oligonucleotide C is synthesized as, for example, four smaller precursor oligonucleotides 97 (oligonucleotides C1 to C4) with C1 containing all of the 40 deoxy thymidine residues. The four precursor oligonucleotides are subsequently ligated, as depicted in FIG. 12B. To create oligonucleotide C, the 5′ ends of signal binding domain oligonucleotides (C2-C4) and the conjugation domain oligonucleotide (C1) are first treated with T4 polynucleotide kinase in the presence of ATP. Oligonucleotides C1-C4 are ligated together with T4 DNA ligase by aligning the oligonucleotides with hydroxy terminated scaffold oligonucleotides 99 of 12 bases in length. The scaffold oligonucleotides 99 are complementary to the 3′ and 5′ ends unique to the junctions of adjacent oligonucleotides, i.e., dA₍₆₎d(CT)₍₃₎; d(CT)₍₃₎:dC₍₆₎; and dC₍₆₎dT₍₆₎ respectively. The central portions (signal binding domains) of oligonucleotides C2 to C4 contain deoxy nucleoside residues that are derivatized with functional groups such as primary amines. Alternatively, functional groups can be introduced into the signal binding domains through incorporation during oligonucleotide synthesis of non-nucleotide compounds that contain functional groups. Phosphoramidites of functional group derivatized deoxy nucleosides or non-nucleoside compounds can be efficiently incorporated into oligonucleotides during chemical synthesis. These phosphoramidites are readily available from commercial sources, e.g., Glen Research (Sterling, Va.) for primary amine derivatized dT phosphoramidite and Clontech (Palo Alto, Calif.) for primary amine derivatized non-nucleoside phosphoramidite called UniLink™ AminoModifier.

After ligation, the reaction mix is heated to 65° C. for 15 minutes to dissociate the scaffold oligonucleotides. The scaffold oligonucleotides and unligated oligonucleotides C1-C4 are relatively small (fewer than 40-50 bases) and therefore, they are readily removed from the ligation product (190 bases) by an appropriate gel filtration column. The quantity and quality of the ligation product are examined by electrophoresis on a polyacrylamide gel followed by, for example, silver staining.

It is understood, however, that the length of oligonucleotide C and the number of functional groups such as primary amines on each oligonucleotide C molecule may vary depending on specific requirements (e.g., sensitivity and linear range etc.) for an assay. It is preferred that the number of derivatized functional groups does not exceed half of the total bases of oligonucleotide C so that the oligonucleotide remains water soluble after conjugated to signal molecules such as acridinium. In some embodiments, oligonucleotide C with fewer than 70, fewer than 60 or fewer than 50 bases contains fewer than 20, fewer than 15 or fewer than 10 functional groups, respectively, but still provides sufficient sensitivity for the assays. In such cases, oligonucleotide C may be a single oligonucleotide that contains both hybridization domain 32 and signal binding domain 34.

### EXAMPLE 3

### Conjugation of Acridinium Ester to Oligonucleotide C

Acridinium has been used in diagnostic assays for many years because of its high quantum yield, which allows its efficient detection and therefore improves the sensitivity of assays. Acridinium is readily available commercially. However, it must be activated to be conjugated to an amine-containing signaling domain. One commonly used method is the introduction of an NHS (N-hydroxysuccinimide) ester to acridinium molecule. One synthesis scheme for making acridinium ester was developed by Week et al, (Clin. Chem. 29: 1474) 1983, incorporated herein by reference, and has been used by several diagnostic manufacturers. Several vendors provide custom synthesis service for acridinium ester, including for example, Molecular Probes (Portland, Oreg.).

Since acridinium succinimidyl ester is reactive with primary amines, acridinium can be conjugated to the signal binding domain 34 of oligonucleotide C through the primary amines introduced to the oligonucleotide as described above. To avoid hydrolysis of NHS esters in water-based solution and therefore improve conjugation efficiency of acridinium to oligo C, the reaction is preferably carried out an organic solvent (e.g., dimethylsulfoxide, DMSO) that is compatible with both oligonucleotides and acridinium ester.

Ten (10) μmoles of acridinium ester (about 5 mg) is dissolved in 1 mL of DMSO and then added to 0.1 μmole oligonucleotide C dissolved in 0.5 mL DMSO. The molar ratio of acridium to primary amine in the oligo is 10 to 1. The mix is incubated 4-5 hours in the dark at room temperature with gentle shaking. After addition of 100 μL of 1 M Tris-HCl, pH 7.5, the mix is incubated under the same conditions for 30 minutes to neutralize un-reacted acridinium ester. The resulted solution is loaded onto a Sephadex G-25 column and eluted with 0.1 M sodium acetate, pH 5.2. The eluent was collected as 0.5 mL fractions and monitored at OD₂₆₀. The peak fractions are collected, mixed together and stored at −70° C.

To estimate the specific activity, 10 μL of the oligonucleotides is diluted to 1.0 mL with 0.1 M Na acetate, pH 5.2. Take 0.5 mL and measure the OD₂₆₀ absorbance, which is used to calculate the oligonucleotide concentration. Take 100 μL and make a series dilution, e.g., 10² to 10¹² fold dilution. Take 50 μL for each dilution and measure the RLU in duplicates. The average RLUs are regressed against the dilution factors, which can be used to estimate the RLU in 50 μL of original (undiluted) solution, which in turn can be used to determine the specific activity, e.g., RLU/pmoles oligo C.

### EXAMPLE 4

### Conjugation of Oligonucleotide Attachment Moieties to Signal Amplification Microparticles and Separating Microparticles

A variety of vendors provide magnetic beads and other forms of microspheres suitable for the present invention. Bangs Laboratories (Fishers, Ind.) offers a variety of microspheric beads of various sizes and materials, including those that are coated with amine functional groups with or without ferromagnetic components. One (1.0) μm magnetic beads are used for the separating microparticle 73 and 1 μm beads are used for the signal amplification microparticle 11. These sizes allow the binding of a suitable analyte, such as a bacterium with one or two magnetic beads and one or two acridinium-labeled signal amplification microparticles 11.

Oligonucleotide attachment moieties 26 and 29 (A and B) both of which contain a primary amine at the 5′ terminus, are conjugated to the signal amplification microparticle 11. Here a method is described that is suitable for conjugating oligonucleotide attachment moieties 26 and 29 to the signal amplification microparticle 11 as well as for conjugating the third oligonucleotide attachment moiety 66 to a magnetic bead comprising the separating microparticle 73. This protocol is adapted from a vendor's protocol (Polysciences, Inc., Warrington, Pa.), which is incorporated herein by reference. Two hundred (200) mg of microparticles derivatized with carboxyl groups (about 240 μmoles/g) is washed twice with reagent grade water using ultrafiltration or centrifugation. The microparticles are suspended in less than 2 mL of water. Immediately prior to use, 384 mg of carbodiimide [1-Ethyl-3-(3-dimethylaminopropyl)-carbodiimide hydrochloride, EDC] is dissolved in 20 mL of 100 mM HEPES buffer (pH 7.5). 1.8 μmoles and 0.2 μmoles of oligonucleotides A and B, respectively, are added to the EDC solution, vortexing vigorously for 2 minutes and making sure that the oligonucleotides is completely dissolved. The resulting oligonucleotide/EDC solution is immediately added to the microparticle solution prepared above. Rotate for 16-24 hours at room temperature. The oligonucleotide-conjugated microparticles are washed three times with 40 mL of water and then suspended in 10 mL of storage buffer containing 1×PBS, 5% glycerol, 1% nuclease free BSA, 0.1% Tween 20, and 0.5% Proclin 5000. Incubate the resulting solution at 68° C. for 4 hours with vigorous shaking every 30 minutes. Finally, rotate the solution for another 16-24 hours at room temperature, replenish the storage buffer and then store at 4-8° C. Oligonucleotide conjugation efficiency is estimated with OD₂₆₀ measurement of the supernate.

### EXAMPLE 5

### Conjugation of Oligonucleotides to Antibodies

Returning to FIG. 12, oligonucleotide D, which has an amine group at the 5′ end, is covalently conjugated to antibodies 41 to form the conjugation domain of the analyte-binding molecule. A preferred method for the conjugation of oligonucleotide D to antibodies is through the use of a heterobifunctional cross linker. One such cross linker, referenced here as an example, is sulfosuccinimidyl 4-(N-maleimidomethyl) cyclohexane-1-carboxylate (Sulfo-SMCC), which consists of an NHS-ester and a maleimide group connected with a spacer arm. Thus, this cross linker can covalently interact with a primary amine on one molecule and with a thio group (sulhydryls) on another molecule, thereby cross-linking the two molecules. If oligonucleotide D contains a primary amine at its terminal, the oligonucleotide is preferably first incubated with sulfo-SMCC at a oligonucleotide to cross linker ratio of 1:5 molar ratio (or empirically determined ratio) for 30-60 minutes at room temperature in PBS buffer. The reaction solution is then passed through a desalt column to remove the un-reacted cross-linker. The resulting oligonucleotide is then incubated with the antibody containing free sulfhydryls, which can be introduced using a reducing agent (e.g., 2-mercaptoethylamine) or through chemical modification (e.g., N-succinimidyl S-acetylthioacetate), at a molar ration of 2:1, which may have to be empirically determined, at 4° C. overnight in PBS buffer. The resulting oligonucleotide-conjugated antibody is separated from unconjugated oligonucleotides through gel filtration chromatography or ultrafiltration using centrifugation. Methods for these separation techniques are well known in the art.

### EXAMPLE 6

### Direct Conjugation of Antibodies to Magnetic Beads

In this alternative, antibodies are attached to the separating microparticle 73 (and/or to the signal amplification microparticle 11) by direct chemical cross linking rather than through a third oligonucleotide attachment moiety 66. This protocol is adapted from a vendor's protocol (Pierce, Rockford, Ill.), which is incorporated herein by reference. Fifty (50) mg of amine-derivatized magnetic beads (240 nmoles amine/mg) are washed three times with 1 mL PBS. The beads are then suspended in 1-mL of PBS, which is added to 1-mL of affinity purified polyclonal antibodies (5 mg/mL). After mixing by gentle agitation, add 0.5 mL of 5 mM freshly prepared BS³. Incubate the mix with gentle agitation at room temperature for 60 minutes. Add 50 μL of 1 M Tris buffer (pH 7.5) and continue the incubation for another 10 minutes to stop the reaction. The microparticle conjugates are separated from unbound antibodies by magnetic separation and washing. The conjugation efficiency is estimated by measuring the absorption of the beads at 280 nm.

### EXAMPLE 7

### Attachment of Acridinium-Labeled Oligonucleotide and Antibodies to Microparticels

Both acridinium-labeled oligonucleotide C and oligonucleotide D labeled antibody 40 are attached to the microparticles through hybridization to oligonucleotides A 26 and B 29, respectively, which are covalently conjugated to the microparticles. To perform hybridization, pre-warmed at 42° C. for 5 minutes the following components: two hundred (200) mg of microparticles coated with oligonucleotides A and B in 5 mL storage buffer that is described in Example 4, 2.0 μmoles of acridinium-labeled oligonucleotide C (5 mL), 22.5 mg (5 mL) of oligonucleotide D conjugated antibody, and 15 mL of 2× hybridization buffer (40 mM Tris-HCl, pH 8.0, 1.0 M NaCl). These components are then mixed together and incubated in a hybridization oven pre-warmed to 42° C. for 2 hours. The mix is then incubated in the dark at room temperature for about 5 hours with rotation. The microparticles are collected and washed four times with PBS using centrifugation or ultrafiltration, and suspended in storage buffer to concentration of 10⁹ particles/mL.

The hybridized double stranded DNA can be further stabilized using UV inducible cross-linking agent such as Psoralen.

### EXAMPLE 8

### Kit Components for Detecting Bacteria in a Platelet Sample

The components and ingredients for detecting are listed in the following table:

TABLE 1

| | Component | Key Ingredient |
|---|---|---|
| 1 | Binding Reagent | Magnetic bead-separating microparticle- |
| | | antibody conjugate at 10⁹ particles/mL |
| | | in storage buffer. |
| 2 | Detection Reagent | Acridinium labeled signal amplification |
| | | microparticles at 10⁹ particles/mL in |
| | | storage buffer. |
| 3 | Negative control | Human plasma without bacteria with |
| | | 12.5 mg/L gentamicin |
| 4 | Positive control | Human plasma with spiked live bacteria |
| | | with 12.5 mg/L gentamicin |
| 5 | Washing Buffer | PBS with 0.01% Tween 20 |
| 6 | Detection buffer A | 0.4 N NaOH |
| 7 | Detection buffer B | 1% hydroperoxide in 0.1 N HC1 |

### EXAMPLE 9

### Optimization of Reaction Conditions for Bacterial Detection Kit

The assay described here is a sandwich format, where the bacteria bind to both magnetic beads and acridinium-labeled microparticles through the same polyclonal antibodies as depicted in FIG. 6.

(i) In order to increase the sensitivity (limit of detection), the concentrations of magnetic beads and microparticles need to be high and/or the incubation time needs to be long to allow formation of a sufficient amount of the complex 65 that is adequate for detection. Since this assay is intended to be a rapid test, incubation times of less than 20 minutes are preferred. The assay is evaluated using 10, 15 or 20-minute incubations. Another variable factor is sample volume and preparation, which can affect the limit of detection, which is defined as the number of bacteria per mL that can be consistently detected (say 95% of the time). However, another design goal for this assay is to make it as simple as possible, which includes little or no sample preparation. Consequently, the present Example is based on use of 1.0 mL of platelets directly taken from the storage medium. For the optimization purpose, human plasma spiked with 5000 bacteria/mL (i.e., the positive control) is used as the optimization sample. Preparation of the antibody and the bacteria has been described in Example 1.

The concentrations of antibody conjugates on the signal amplification microparticle and the separating microparticle, the ratio of these two conjugates in the test procedure, are initially evaluated using an incubation time (10 minutes) and a sample of 1.0-mL positive without further preparation. The determination of the ratio for the two conjugates would be to identify the ratio that gives the highest signal to cutoff ratio. The combinations of the two conjugates used in the initial test are listed in Table 2

TABLE 2
Optimization for the concentrations of conjugates

| | | | Antibody- | | |
|---|---|---|---|---|---|
| | | | oligo/acridinium | | |
| | | Antibody- | microparticle | | |
| | | Magnetic bead | conjugates | | |
| | | conjugates | (signal | | |
| | | (separating | amplification | RLU (relative | |
| RXN | Sample | microparticles) | microparticles) | light unit) | S/CO |
| In | 1.0-mL | 10⁶ particles | 10⁶ particles | | Not |
| | negative control | | | | relevant |
| 2n | 1.0-mL | 10⁶ particles | 10⁷ particles | | Not |
| | negative control | | | | relevant |
| 3n | 1.0-mL | 10⁶ particles | 10⁸ particles | | Not |
| | negative control | | | | relevant |
| 4n | 1.0-mL | 10⁷ particles | 10⁶ particles | | Not |
| | negative control | | | | relevant |
| 5n | 1.0-mL | 10⁷ particles | 10⁷ particles | | Not |
| | negative control | | | | relevant |
| 6n | 1.0-mL | 10⁷ particles | 10⁸ particles | | Not |
| | negative control | | | | relevant |
| 7n | 1.0-mL | 10⁸ particles | 10⁶ particles | | Not |
| | negative control | | | | relevant |
| 8n | 1.0-mL | 10⁸ particles | 10⁷ particles | | Not |
| | negative control | | | | relevant |
| 9n | 1.0-mL | 10⁸ particles | 10⁸ particles | | Not |
| | negative control | | | | relevant |
| 1p | 1.0 mL Positive | 10⁶ particles | 10⁶ particles | | |
| | control | | | | |
| 2p | 1.0 mL Positive | 10⁶ particles | 10⁷ particles | | |
| | control | | | | |
| 3p | 1.0 mL Positive | 10⁶ particles | 10⁸ particles | | |
| | control | | | | |
| 4p | 1.0 mL Positive | 10⁷ particles | 10⁶ particles | | |
| | control | | | | |
| 5p | 1.0 mL Positive | 10⁷ particles | 10⁷ particles | | |
| | control | | | | |
| 6p | 1.0 mL Positive | 10⁷ particles | 10⁸ particles | | |
| | control | | | | |
| 7p | 1.0 mL Positive | 10⁸ particles | 10⁶ particles | | |
| | control | | | | |
| 8p | 1.0 mL Positive | 10⁸ particles | 10⁷ particles | | |
| | control | | | | |
| 9p | 1.0 mL Positive | 10⁸ particles | 10⁸ particles | | |
| | control | | | | |
| 10p | 1.0 mL Positive | 10⁸ particles | None | | |
| | control | | | | |
| 11p | 1.0 mL Positive | None | 10⁸ particles | | |
| | control | | | | |

The signal to cutoff ratio (S/CO) is the value resulted from the RLU of positive control for a given condition divided by the RLU of negative control for the same condition. The particle counts are determined according to the information on Certificate of Analysis provided by the vendor assuming a 85% recovery rate after the conjugation processes.

The signal to cutoff ratio (S/CO) is the value resulted from the RLU of positive control for a given condition divided by the RLU of negative control for the same condition. The particle counts are determined according to the information on Certificate of Analysis provided by the vendor assuming a 85% recovery rate after the conjugation processes.

(ii) Initial Optimization Protocol. The following protocol is to be used for conjugate optimization as indicated in table 2.

(a) Supplies: Magnet, reaction tubes (75×12 mm glass tubes for scintillation counter or other similar-size transparent tubes), and kit components as listed in Table 1 in Example 8.

(b) Take 20 reaction tubes and label them with the number indicated in table 2. Add indicated amounts (see Table 2) of magnetic bead conjugates and acridinium conjugates

(c). Add 1.0 mL of positive control to reactions 10p-20p and 1.0 mL of negative control to reactions 1n-9n. Mix and incubate at room temperature with gentle agitation in a rotator for 10 minutes.

(d). Place the tubes near a magnetic bar designed for these tubes. Waite for two minutes or until all magnetic beads are attached to the tube wall.

(e). Decant all solution in the reaction tubes. Remove the last drop of solution by touching the tube lip on a piece of Kimwipe paper or other absorptive paper.

(f). While still keeping the tubes on the magnetic bar, add 5-mL of wash buffer and repeat steps d and e). Repeat this step one more time.

(g). Read the results with the luminometer. Record the RLU in Table 2.

(h). Calculate the S/CO for each conjugate.

This procedure will allow the identification of an optimal (or near optimal) combination of the two conjugates. A similar protocol is used to optimize the antibody to signaling molecule on the signal amplification microparticle. In that procedure, the ratio of separating microparticles to signal amplification microparticles is fixed in groups, while the ratio of antibody conjugate to signaling molecule conjugate is varied within each group. It may be necessary to repeat this experiment twice or more to ascertain the reproducibility of the test results.

A luminometer with two injectors is suitable for the assay.

(iii) Further optimization for conjugate concentrations (optional). There are only three conjugate concentration levels for each conjugate but they differ by 10 folds. It is likely that the “optimal” concentrations can be further optimized. An indication that none of the combinations is optimal is that more than one conjugate concentration combinations gives similarly high S/CO. In this case, conjugate concentrations around the peaks are further optimized.

(iv) Optimization for sample amounts and preparation (optional). If none of the conjugate combinations gives a reasonably high S/CO (e.g., <2.0), which may indicate that the bacteria concentration is too low. To “increase” the concentration of bacteria, the samples (1.0 mL) are centrifuged at 6,000 rpm for 5 minutes in a microfuge to pellet the bacteria. Remove 0.9 mL of supernatant and suspend the cell pellet in the remaining solution. This step results in roughly 10-fold concentration increase and may make a significant difference in terms of limit of detection. However, this step adds about 5 minutes to the procedure, but incubation time may be decreased to less than 10 minutes. Of course, more than 1 mL of sample can be used if a centrifugation step is involved in the sample preparation.

(v) Optimization for incubation time. The incubation time is incrementally increased in 5 minute increments to at least 15 or 20 minutes or the longest incubation time desired for a given test. In general, increasing incubation time will increase analyte binding until the point where the system reaches equilibrium, which is in turn determined by the affinity constants of the analyte binding molecules.

(vi) Optimization for background RLU (negative control). The background RLU is expected to be very low since signal molecules are directly conjugated to the signal amplification microparticles, which can be easily washed away. However, the two “washing” steps in the protocol are in fact “rinsing” steps, which may not completely remove unbound microparticles and thus result in higher background. If the background does appear to be high (e.g., several fold over the RLU without acridinium-labeled microparticles), the number of washing steps is increased. This modification may add another 5 minutes to the procedure. If, on the other hand, the background were similar to the RLU of a control without acridinium-labeled microparticles, it would indicate that the “rinsing” step is effective in removing unbound microparticles. Each deletion of a rinsing step saves 3-4 minutes for the procedure. A preferred procedure uses only one rinsing step.

(vii) Alternate component formation. A kit may be simplified to include a single component that contains the signal amplification microparticles 11 and the separating microparticles 73. The viability of such a formulation will depend on the long-term stability of such a formulation, which is assessed by making such a kit and testing results with a positive control over time.

(viii) Reproducibility study—setting the cutoff and determining limit of detection. Once the assay is optimized for maximal S/CO, the variability of the assay is assessed by conducting a reproducibility study to obtain data for setting a positive (or negative) S/CO value and determining the limit of detection.

(ix) The protocol. One set of test runs are conducted each day over five days, which are not necessarily consecutive days. Each day, a bag of platelets is obtained. A portion of it is removed for plate culture using plates to confirm that it is free from bacterial contamination. Remove 50 mL as negative sample. Remove another 5×15-mL (60 mL) and place them in five sterile tubes, to which the positive control bacteria are spiked at a final bacterial concentration of 1000, 2000, 4000, 6000 and 8000/mL, respectively. Forty and 10 replicates are tested for the negative and positive samples each day, respectively. To increase the number of “runs”, these replicates may be broken into several mini-runs each day. For example, a run may test 8 negative sample replicates and 2 replicates for each positive sample; such, five mini-runs are performed each day. RLU values are obtained for each test. Upon completion of this study, there are 200 test results for the negative samples and 50 for each positive sample.

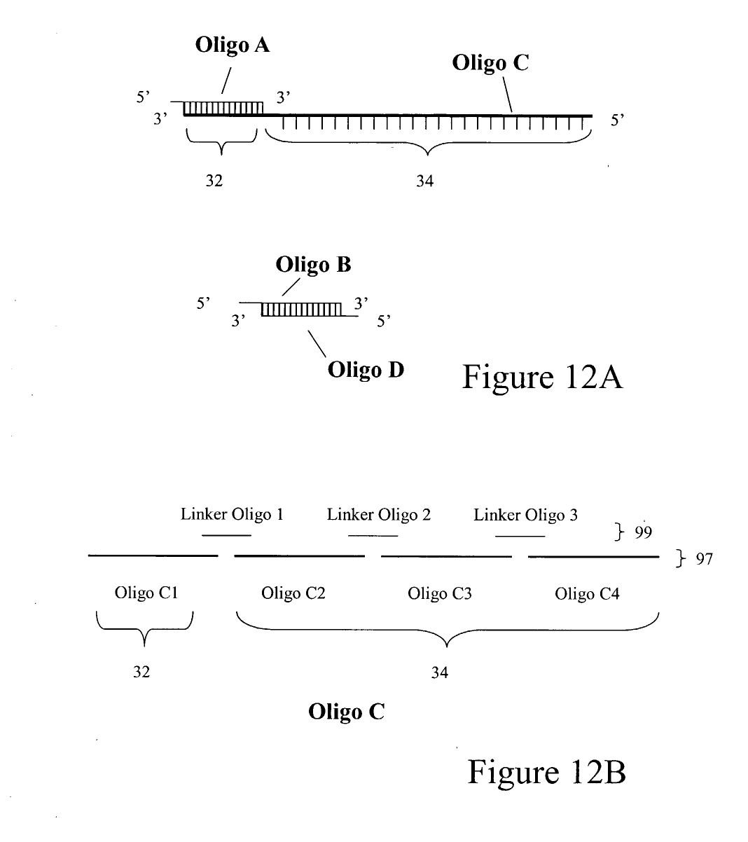

(x) A standard deviation (SD) and mean of the RLU values for negative control are calculated from the 200 test results (RLU values) of the negative control. A cutoff RLU for negativity (or positivity) is set according to required specificity. For example, a specificity of 99% is achieved if the cutoff RLU is set at 3 SD above the mean, as depicted in FIG. 13. This cutoff RLU is used as denominator to divide the RLU of a sample to generate a S/CO (signal to cutoff ratio) for the sample. Thus, if the S/CO is equal to or greater than 1.0, the sample is considered positive.

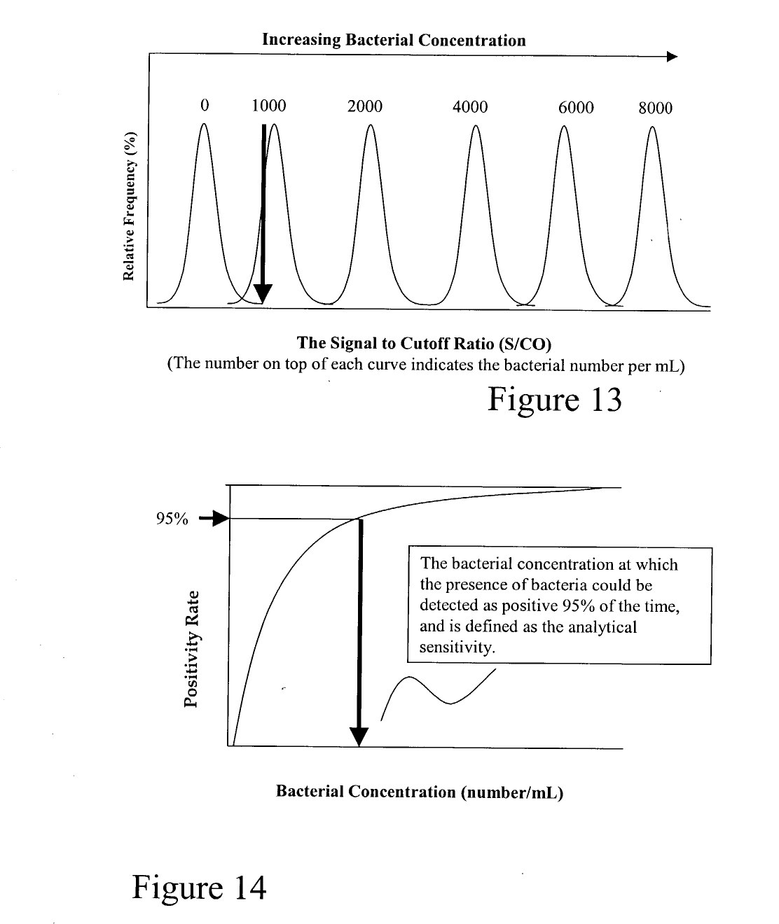

(xi) Estimation of limit of detection (analytical sensitivity). Once the S/CO for positivity is set (e.g., =1.0), one is able to determine whether a test for bacteria-containing sample is positive, from which one can calculate the percentage of positive results for each bacteria concentration level. The rate of positivity for each bacteria concentration is then used to estimate the limit of detection using “Probit Modeling” statistics method as described by Finney, D. J. (1971) (Probit Analysis, Third Edition, London: Cambridge University Press), incorporated here as a reference. Software programs for Probit analysis are available from SAS Institute Inc. (Cary, N.C.). The limit of detection is determined as the bacteria level where the sample can be tested positive 95% of the time as depicted in FIG. 14.

(xii) Detection of bacteria growing in platelets. A unit of freshly prepared platelets is spiked with 5000 bacteria (E. coli, M15). To ensure that no other strain of bacteria is growing in the platelet, kanamycin is added to a final concentration of 25 μg/mL. Four mL of platelets are removed about every 12 hours, 1 mL is used for a plate culture to determine the bacterial concentration while two mL are used for testing in duplicates following the optimized protocol. This study evaluates the performance of the assay for bacterial grown under simulated conditions.

### EXAMPLE 10

### An HIV-1 Viral Load Test Using Microparticle Based Amplification

As depicted in FIG. 10, this invention can be applied to qualitative or quantitative detection of nucleic acids. This example describes one method for the quantitative detection of HIV-1 virus, e.g., the determination of viral concentration in a sample.

Sequence selection and synthesis of oligonucleotides. The assay requires at least one pair, at least two pairs, at least three pairs or at least four pairs of oligonucleotides, which, or a portion of which, are complementary to highly conserved regions of HIV-1 sequences. Methods for selecting conserved sequences are well known to those of ordinary skill in the art. The two oligonucleotides in a pair are preferred, but not necessarily complementary to the same region of (but distinct sequences in) the viral genome. One of the oligonucleotides in a pair is conjugated to separating microparticle 73 while the other oligonucleotide is linked to signal amplification microparticle 11. The conjugation of these oligonucletotides to microparticles can be direct conjugation as depicted in FIG. 3A, or indirect via a third molecule as depicted in FIGS. 3B and 3C.

In a design with four pairs of oligonucleotides, the separating microparticle 73 and signal amplification microparticle 11 will each be conjugated with four oligonucleotides. The length of the oligonucleotides should be sufficiently long to provide the DNA/RNA hybrid with a melting point of at least 40° C., at least 50° C., or at least 60° C. The oligonucleotides are synthesized with derivatized functional groups (e.g., primary amines) at their 3′ or 5′ terminus. As illustrated in FIG. 10, the signal amplification microparticles 11 contains two additional oligonucleotides, the signal molecule 31 and first attachment oligonucleotide 26, the latter being complementary to the binding domain 32 of signal molecule 31. Preparation of signal molecule 31 and first attachment oligonucleotide 26 were described in Examples 2 and 3.

Attachment of oligonucleotides to microparticles. Oligonucleotides are attached to microparticle 10 and 60 to form signal amplification microparticles 11 and separating microparticles 73, respectively. One method for attaching oligonucleotides to microparticles is described in Example 4 and can be used for manufacturing the HIV-1 assay. The molar ratio of first attachment oligonucleotide 26 to viral specific oligonucleotides, which are complementary to viral genome, should be determined empirically, but one can start with, for example, a ratio of 1:4. Equal moles of viral specific oligonucleotides are preferred for the attachment to both microparticles 10 and 60. Once viral specific oligonucleotide(s) are conjugated to microparticles 60, the microparticles become functional separating microparticles 73, which are suspended in storage buffer at 10⁹ particles/mL.

The assembly of functional signal amplification microparticles 11 requires the attachment of signal molecules 31 through hybridization, as described in general in Example 7. Specifically, the following components are pre-warmed at 42° C. for 5 minutes: two hundred (200) mg of microparticles coated with oligonucleotides (5 mL), 2.0 μmoles of acridinium-labeled oligonucleotide C (5 mL), and 10 mL of 2× hybridization buffer (40 mM Tris-HCl, pH 8.0, 1.0 M NaCl). These components are then mixed together and incubated in a hybridization oven pre-warmed to 42° C. for 2 hours. The mix is then incubated in the dark at room temperature for about 5 hours with rotation. The microparticles are collected and washed four times with PBS using centrifugation or ultrafiltration, and suspended in storage buffer at 10⁹ particles/mL.

Sample preparation. The clinical samples are preferred to be human plasma collected from individuals for HIV-1 viral load testing using anticoagulant such as heparin and EDTA. After removal of red cells and white cells, and the like, by low speed centrifugation (e.g., 3000 g for 10 minutes), the resulting plasma is used for viral load testing. It is preferred that the ribonucleic acids (RNA) in the samples are first extracted from the plasma. Methods for extracting RNA from clinical samples are well known to those of ordinary skill in the art. Commercial sources (e.g., Invitrogen, San Diego, Calif.) of RNA extraction solutions are available. The RNA extract can be suspended in 100 μL of TE buffer (10 mM Tris-HCl, pH 8.0 and 0.5 mM EDTA).

Test protocol. Add 300 μL of 2× hybridization buffer, 100 μL of separating microparticles and 100 μL of signal amplification microparticles to the RNA sample (100 μL). Incubate at 42° C. for 3 hours with gentle agitation. Transfer the reaction solution to a 12×75 mm test tube, which is then placed near a magnet. After the separating microparticles are attracted to the test tube wall (approximately 2-3 minutes), the aqueous solution is removed. Wash the microparticles four times with 2 mL of PBS buffer. Suspend the microparticles in 100 μL of PBS buffer. Measure the light output after addition of 300 μL of 1% hydroperoxide in 0.1 N HCl and 100 μL of 0.4 N NaOH. The viral RNA concentration in the sample is determined using a linear curve, which may be predetermined or concurrently determined.

Optimization of the assay. Assay conditions that may have to be optimized include, but are not limited to, the amounts of separating microparticles and signal amplification particles, acridinium labeling intensity on signal amplification microparticles, hybridization conditions (e.g., salt concentration, duration and temperature), and the numbers of washing cycles. One method for optimizing the relative amounts of separating and signal amplification microparticles is described in Example 9 and is applicable to the HIV viral load assay. Acridinium labeling intensity should be optimized in a quantitative assay since optimal intensity allows the assay to have a wider linear range. Accordingly, the optimal acridinium labeling intensity is determined in a linearity study such that a desired linear range (e.g, 100-50,000 HIV-1 RNA copies/mL) is achieved. Optimal hybridization conditions are those that allow sufficient hybridization in the shortest time. The optimal number of wash cycles is the least number of cycles that result in lowest background.

Establishment of a standard curve. For a quantitative assay, a standard curve is normally, but not necessarily, established for each run by testing a series of samples with increasing known concentrations of HIV-1 viral RNA, e.g., from 0 to 50,000 copies/mL. The resulting RLU outputs are regressed against the viral concentrations to generate a linear curve. This linear curve is used to calculate HIV-1 RNA concentration in the samples.

An alternative method is to establish a standard curve for the assay, not for a specific run, by testing a large number of standards under a variety of situations, e.g., different laboratories and reagent lots. To control run specific variation, a few calibrators (e.g., three) with known concentrations of HIV-1 RNA may be tested in each run to calibrate the standard curve for the run.

Determination of assay performance. Several key parameters indicative of assay performance may be characterized. They include, but are not limited to, linear range (upper and lower quantitative limits), linearity, and precision. Methods for evaluating linearity and precision, such as those described in the NCCLS (National Committee for Clinical Laboratory Standards) guidance documents, are well known in the art, and may be used for evaluating this assay.

1. A microparticle for detecting an analyte comprising,
a signal amplification microparticle that includes a signal molecule comprising a first conjugation domain bound to the microparticle and a signal binding domain bound to a plurality of signal emitting moieties; and
an analyte binding molecule comprising an analyte binding domain that binds the analyte and a second conjugation domain bound to the microparticle.

2. The microparticle of claim 1 wherein at least one of the first conjugation domain and the second conjugation domain is cross linked to a functional group on the surface of the signal amplification microparticle.

3. The microparticle of claim 1 wherein the first conjugation domain is bound to the signal amplification microparticle through a first attachment moiety cross linked to a functional group on the surface of the signal amplification microparticle and wherein the second conjugation domain is bound to the signal amplification microparticle through a second attachment moiety cross linked to a functional group on the surface of the signal amplification microparticle.

4. The microparticle of claim 3 wherein at least one of the first attachment moiety and the second attachment moiety is comprised of an oligonucleotide.

5. The microparticle of claim 3 wherein each of the first attachment moiety and the second attachment moiety is comprised of an oligonucleotide.

6. The microparticle of claim 3 wherein at least one of the first attachment moiety and the second attachment moiety is comprised of a first oligonucleotide sequence and wherein at least one of the first conjugation domain and the second conjugation domain, respectively, is comprised of a second nucleotide sequence complementary to the first oligonucleotide sequence.

7. The microparticle of claim 3 wherein the first attachment moiety is comprised of a first oligonucleotide sequence, the first conjugation domain is comprised of a second nucleotide sequence complementary to the first oligonucleotide sequence, the second attachment moiety is comprised of a third oligonucleotide sequence and the second conjugation domain is comprised of a fourth nucleotide sequence complementary to the third oligonucleotide sequence.

8. The microparticle of claim 7 wherein the first oligonucleotide sequence is different than the third oligonucleotide sequence.

9. The microparticle of claim 7 wherein the first oligonucleotide sequence is the same as the third oligonucleotide sequence.

10. The microparticle of claim 1 wherein the signal binding domain of the signal molecule is comprised of a polymer selected from the group consisting of a polynucleotide, polylysine, polyarginine, polyglutamine, polyhistidine, a poly-amino-saccharides, spermine, spermidine, and a polypeptides having a plurality of amine side-chain functional groups.

11. The microparticle of claim 10 wherein the signal molecule is further comprised of a first nucleic sequence that forms the first conjugation domain that binds the first attachment moiety, the first attachment moiety being comprised of an oligonucleotide sequence complimentary to the first nucleic acid sequence.

12. The microparticle of claim 1 wherein the signal molecules is comprised of a first nucleic sequence that forms the first conjugation domain that binds the first attachment moiety, the first attachment moiety being comprised of an oligonucleotide sequence complimentary to the first nucleic acid sequence, and wherein the signal molecule is further comprised of a second nucleic acid sequence that forms the signal binding domain.

13. The microparticle of claim 1 wherein the signal binding domain of the signal molecule is comprised of a polymer selected from the group consisting of polyglutamate, polyasparte, polyglyconate and polypeptides having a plurality of carboxyl side-chain functional groups.

14. The microparticle of claim 13 wherein the signal molecule is further comprised of a first nucleic sequence that forms the first conjugation domain that binds the first attachment moiety, the first attachment moiety being comprised of an oligonucleotide sequence complimentary to the first nucleic acid sequence.

15. The microparticle of claim 1 wherein the plurality of signal emitting moieties are selected from the group consisting of a chemiluminescent moiety, a phosphorescent moiety, an electroluminescent moiety and a fluorescent moiety.

16. The microparticle of claim 15 wherein the plurality of signal emitting moieties do not emit a signal under a first condition and does emit the signal under a second condition different than the first condition.

17. The microparticle of claim 16 wherein at least one of the first condition and the second condition is an oxidizing condition relative to the other of the second condition and the first condition, respectively.

18. The microparticle of claim 1 wherein the plurality of signal emitting moieties are comprised of acridinium residues.

19. The microparticle of claim 1 wherein the signal binding domain is comprised of a nucleic acid sequence and the plurality of signal emitting moieties are comprised of acridinium residues bound to the nucleic acid sequence of the signal binding domain.

20. The microparticle of claim 1 wherein the plurality of signal emitting moieties are comprised of fluorescein residues.

21. The microparticle of claim 1 wherein the signal binding domain of the signal molecule is bound to at least 10 signal emitting moieties per signal molecule.

22. The microparticle of claim 1 wherein the signal binding domain of the signal molecule is bound to at least 50 signal emitting moieties per signal molecule.

23. The microparticle of claim 1 wherein the signal binding domain of the signal molecule is bound to at least 100 signal emitting moieties per signal molecule.

24. The microparticle of claim 1 wherein the signal binding domain of the signal molecule is bound to at least 150 signal emitting moieties per signal molecule.

25. The microparticle of claim 1 wherein the analyte binding molecule is comprised of an antibody that binds the analyte, the analyte being comprised of an antigen.

26. The microparticle of claim 25 wherein the antibody is bound to an oligonucleotide, the oligonucleotide forming the second conjugation domain.

27. The microparticle of claim 25 wherein the antibody is comprised of a polyclonal antibody.

28. The microparticle of claim 25 wherein the antibody is comprised of a monoclonal antibody.

29. The microparticle of claim 1 wherein the analyte binding molecules is comprised of an antigen that binds the analyte, the analyte being comprised of an antibody.

30. The microparticle of claim 29 wherein the antigen is bound to an oligonucleotide, the oligonucleotide forming the second conjugation domain.

31. The microparticle of claim 29 wherein the antigen is conjugated to a carrier molecule and the carrier molecule is bound to an oligonucleotide, the oligonucleotide forming the second conjugation domain.

32. The microparticle of claim 1 wherein the analyte binding molecule is comprised of a first nucleic acid sequence that binds to the analyte, the analyte being comprised of a target nucleic acid sequence that is complementary to the first nucleic acid sequence.

33. The microparticle of claim 32 wherein the analyte binding molecule is further comprised of a second nucleic sequence, the second nucleic acid sequence forming the second conjugation domain.

34. The microparticle of claim 1 wherein a ratio of the signal molecules to the analyte binding molecules is at least 3 to 1.

35. The microparticle of claim 1 wherein a ratio of the signal molecules to the analyte binding molecules is at least 10 to 1.

36. The microparticle of claim 1 wherein the microparticle has a diameter of less than about 2 micrometers.

37. The microparticle of claim 1 wherein the microparticle has a diameter of less than about 1 micrometer.

38. The microparticle of claim 1 wherein the microparticle has a diameter of about 0.001 micrometer to about 1.0 micrometer.

39. The microparticle of claim 1 wherein the microparticle has a diameter of about 0.01 to 0.5 micrometer.

40. A microparticle for detecting an analyte comprising,
microparticle of less than about 2 μm in diameter that includes,
a plurality of signal molecules bound to the microparticle, each signal molecule comprising a first oligonucleotide conjugation domain that is bound to the microparticle through a first oligonucleotide attachment moiety attached to the microparticle, the signal molecule further including a signal binding domain comprised of a plurality of functional groups bound to a plurality of acridinium moieties; and
a plurality of analyte binding molecules bound to the microparticle, each analyte binding molecule comprising an analyte binding domain that binds the analyte and a second oligonucleotide conjugation domain that is bound to the signal amplification microparticle through a second oligonucleotide attachment moiety attached to the microparticle.

41. A kit for detecting an analyte comprising,
a first microparticle according to claim 1; and
a second microparticle having a physical property that is distinct from the first microparticle, the second microparticle being bound to a second analyte binding molecule having a third conjugation domain which is bound to the second microparticle and the second analyte binding molecule having a second analyte binding domain that binds the analyte.

42. The kit of claim 41 wherein the third conjugation domain is cross-linked to a functional group on the surface of the second microparticle.

43. The microparticle of claim 41 wherein the third conjugation domain is bound to the second microparticle through a third attachment moiety cross linked to a functional group on the surface of the second microparticle.

44. The kit of claim 43 wherein the third attachment moiety is comprised of an oligonucleotide sequence and the third conjugation domain is comprised of a nucleotide sequence that is complementary to the oligonucleotide sequence of the third attachment moiety.

45. The kit of claim 41 wherein the physical property of the second microparticle permits separation of the second microparticle from the first microparticle unless the first microparticle is associated with the second microparticle by cross binding of the analyte.

46. The kit of claim 41 wherein the physical property of the second microparticle includes a larger size than the first microparticle.

47. The kit of claim 41 wherein the physical property of the second microparticle comprises a ferromagnetic property the first microparticles being non-ferromagnetic.

48. The kit of claim 41 wherein the physical property of the second microparticle comprises fluorescence of a molecule bound to the second microparticle.

49. The kit of claim 41 wherein the first analyte binding domain binds a different region of the analyte than the second analyte binding domain.

50. The kit of claim 1 wherein at least one of the first and the second analyte binding molecules is an antigen.

51. The kit of claim 1 wherein at least one of the first and the second analyte binding molecules is an antibody.

52. The kit of claim 20 wherein each the first and the second analyte binding molecules is an antibody.

53. The kit of claim 1 wherein at least one of the first and the second analyte binding molecules is a nucleic acid.

54. The kit of claim 1 wherein at each of the first and the second analyte binding domains is comprised of a nucleic acid sequence and where the first analyte binding domain includes a first nucleic acid sequence complementary to a first target sequence on the analyte and the second nucleic acid sequence includes a second nucleic acid sequence complementary to a second target sequence on the analyte binding molecule.

55. A method for detecting an analyte comprising,
contacting a sample containing the analyte with a first microparticle according to claim 1;
contacting the sample with a second microparticle having a physical property that is different from the first microparticle, the second microparticle being bound to a second analyte binding molecule having a third conjugation domain that is bound to the second microparticle, and the second analyte binding molecule having a second analyte binding domain that binds the analyte.
incubating the sample for a period of time sufficient to form a bound complex comprised of the first microparticle and the second microparticle, each bound to the analyte;
separating a first fraction from the sample containing the bound complex from a second fraction containing the first microparticles not bound in the complex;
retaining the first fraction; and
detecting an amount of signal emitted from the signal emitting moieties in the first fraction.

56. The method of claim 55 further including washing the first fraction containing the bound complex to remove a portion of first microparticles associated with the first fraction other than by being bound in the complex, and then repeating the act of separating.

57. The method of claim 55 wherein the physical property of the second microparticle is a ferromagnetic property and wherein the act of separating includes attracting the bound complex toward a magnet.

58. The method of claim 55 wherein the physical property of the second microparticle is a fluorescent property, and wherein the act of separating includes separating the bound complex through a fluorescence activated cell sorter.

59. The method of claim 55 wherein the physical property of the second microparticle is a larger size than the first microparticle, and wherein the act of separating includes recovering the bound complex by at least one of filtration or centrifugation.

60. The method of claim 55 wherein the period of time of incubation is about 15 minutes or less.

61. The method of claim 55 wherein the period of time of incubation is about 10 minutes or less.

62. The method of claim 55 wherein the period of time of incubation is about 5 minutes or less.

63. The microparticle of claim 1 further including a vehicle molecule wherein the signal molecule and the analyte binding molecule are bound to the vehicle molecule and the vehicle molecule is bound to the microparticle, and wherein the vehicle molecule includes a vehicle molecule attachment moiety that comprises both the first and the second conjugation domains of the signal molecule and the analyte binding molecule, respectively.

64. The microparticle of claim 63 wherein the first vehicle molecule attachment moiety is a nucleic acid sequence that binds a complementary nucleic acid sequence that comprises a second attachment moiety which is bound to the microparticle.

65. The microparticle of claim 63 wherein the vehicle molecule is comprised of polylysine.

66. The microparticle of claim 65 wherein the signal binding domain of the signal molecule is comprised of the polylysine.

67. The microparticle of claim 63 wherein the first conjugation of the signal molecule is comprised of an oligonucleotide that is bound to the polylysine through a complementary oligonucleotide.

68. The microparticle of claim 25 wherein the antibody binds an HIV antigen.

69. The microparticle of claim 32 wherein the target nucleic acid sequence is a nucleic acid sequence encoded by an HIV.

70. The microparticle of claim 69 further comprising a second analyte binding molecule, the second analyte binding molecule comprised of a second nucleic acid sequence complementary to a second target sequence encoded by an HIV, the first target sequence being different from the first target sequence.

71. The kit of claim 54, wherein at least one of the first and second target sequences are nucleic acid sequence encoded by an HIV.

72. The kit of claim 54, wherein each of the first and second target sequences are nucleic acid sequence encoded by an HIV, the first and second target sequences being different.

73. A microparticle for detecting an analyte comprising,
a signal amplification microparticle that includes a signal molecule comprising a first conjugation domain bound to the microparticle and a signal binding domain bound to a signaling molecule; and
an analyte binding molecule comprising an analyte binding domain that binds the analyte and a second conjugation domain bound to the microparticle.

74. The microparticle of claim 73 wherein the signaling molecule comprises an enzyme that converts a substrate into a product.

75. The microparticle of claim 73 wherein the signaling molecule comprises acridinium and the signal binding domain is bound a plurality of acridinium moieties.
